(19) United States
(12) Patent Application Publication
(10) Pub. No.: US 2004/0167074 A1
(43) Pub. Date: Aug. 26, 2004

(54) Therapeutic applications of mFLINT polypeptides

(76) Inventors: Thomas Frank Bumol, Carmel, IN (US); Shenshen Dou, Carmel, IN (US); Andrew Lawrence Glasebrook, Zionsville, IN (US); Songqing Na, Carmel, IN (US); Ho Yeong Song, Carmel, IN (US); Steven Harold Zuckerman, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/793,269

(22) Filed: Mar. 4, 2004

Related U.S. Application Data
(62) Division of application No. 09/280,567, filed on Mar. 30, 1999.
(60) Provisional application No. 60/079,856, filed on Mar. 30, 1998.
(60) Provisional application No. 60/086,074, filed on May 20, 1998.
(60) Provisional application No. 60/099,643, filed on Sep. 9, 1998.
(60) Provisional application No. 60/112,703, filed on Dec. 18, 1998.
(60) Provisional application No. 60/112,577, filed on Dec. 17, 1998.
(60) Provisional application No. 60/112,933, filed on Dec. 18, 1998.
(60) Provisional application No. 60/113,407, filed on Dec. 22, 1998.

(51) Int. Cl. A61K 38/17

(52) U.S. Cl. 514/12

(57) ABSTRACT

Mature FLINT protein (mFLINT) binds FasL and LIGHT, and prevents FasL-Fas interaction. mFLINT inhibits FasL-Fas-mediated apoptotic and proinflammatory activity, and is useful in treating disorders associated with abnormal apoptosis and inflammation. The invention provides the amino acid and nucleotide sequences of FLINT and mature FLINT. The preparation and characterization of transgenic animals that express FLINT is disclosed. Therapeutic compositions and methods of treatment utilizing mFLINT also are provided.

This application claims priority to the following U.S. provisional patent applications: 60/079,856, filed Mar. 30, 1998; 60/086,074, filed May 20, 1998; 60/099,643, filed Sep. 9, 1998; 60/112,703, filed Dec. 18, 1998; 60/112,577, filed Dec. 17, 1998; 60/112,933, filed Dec. 18, 1998; and 60/113,407, filed Dec. 22, 1998.

### BACKGROUND OF THE INVENTION

FasL (also called CD95L and APO1L) is expressed on various cell types and can produce biological responses such as proliferation, differentiation, immunoregulation, inflammatory response, cytotoxicity, and apoptosis. Interestingly, mutations in FasL, the ligand for the TNFR-family receptor FAS/APO (Suda et al., 1993, Cell 75:1169-78, are associated with autoimmunity (Fisher et al., 1995, Cell 81:935-46), while overproduction of FasL may be implicated in drug-induced hepatitis. FasL is expressed in immune-privileged tissues of the eye, testis, brain and some tumors. It has also been found in kidney and lung as well as in activated thymocytes, splenocytes, and T lymphocytes.

Apoptosis plays a central role in both development and in homeostasis. Cells die by apoptosis in the developing embryo during morphogenesis or synaptogenesis and in the adult animal during tissue turnover or at the end of an immune response. Because the physiological role of apoptosis is crucial, aberration of this process can be detrimental. For example, unscheduled apoptosis of certain brain neurons contributes to disorders such as Alzheimer's and Parkinson's disease, whereas the failure of dividing cells to initiate apoptosis after sustaining severe DNA damage contributes to cancer.

Survival signals from the cell's environment and internal sensors for cellular integrity normally keep a cell's apoptotic machinery in check. In the event that a cell loses contact with its surroundings or sustains irreparable damage, the cell initiates apoptosis. A cell that simultaneously receives conflicting signals driving or attenuating its division cycle also triggers apoptosis. Mammals have evolved yet another mechanism that enables the organism actively to direct individual cells to self-destruct. This kind of “instructive” apoptosis is important especially in the immune system. Death receptors-cell surface receptors that transmit apoptosis signals initiated by specific “death ligands”—play a central role in instructive role apoptosis. These receptors can activate death caspases within seconds of ligands binding, causing an apoptotic demise of the cell within hours.

Death receptors belong to the tumor necrosis factor (TNF) receptor gene superfamily, which is defined by similar, cysteine-rich extracellular domains. The death receptors contain in additional a homologous cytoplasmic sequence termed the “death domain”. Death domains typically enable death receptors to engage the cell's apoptotic machinery, but in some instances they mediate functions that are distinct from or even counteract apoptosis.

Fas (also called CD95 or Apo1) is a well-characterized death receptor. Fas and Fas ligand (FasL) play an important role in apoptosis. Fas L is a homotrimeric molecule. It is suggested that each FasL timer binds three Fas molecules. Because death domains have a propensity to associate with one another, Fas ligation leads to clustering of the receptors' death domains. An adapter protein called FADD (Fas-associated death domain; also called Mort1) then binds through its own death domain to the clustered receptor death domains. FADD also contains a “death effector domain” that binds to an analogous domain repeated in tandem within the zymogen form of caspase-8 (also called FLICE, or MACH). Upon recruitment by FADD, caspase-8 oligomerization drives its activation through self-cleavage. Caspase-8 then activates downstream effector caspases such as caspase-9 committing the cell to apoptosis. Ashkenazi A., et al. “Death Receptors: Signaling and Modulation Science 281, 1305-1308 (August 1998).

Although it triggers apoptosis in T lymphocytes, FasL is also proinflammatory. FasL has been shown to stimulate neutrophils, also called polymorphonuclear leukocytes (PMNs), activation. (Chen J. et at, Science 282: 1714-17 (1998)) FasL-Fas binding has been implicated in clonal deletions of autoreactive lymphocytes in peripheral lymphoid tissues and in elimination of autoreactive lymphocyte populations, thus contributing to homeostasis of the immune system. However, it has been found that expression of FasL on myotubes or pancreatic islets of transgenic mice induces a granulocytic response that accelerates graft rejection (Allison J. et al., Proc. Natl. Acad. Sci, 94:3943-47 (April 1997); Kang S-M. et al., Nature Medicien, Vol. 3, No. 7, 738-743 (July 1997)).

At least one of the effects of FasL-Fas receptor binding is apoptosis, which is necessary for homeostasis. However, sometimes the balance of ligand-receptors binding is upset in stress, disease or trauma. One of the negative effects of unregulated FasL-Fas binding is runaway or aberrant apoptosis. Another effect of said binding is the destruction of healthy cells caused by neutrophils that have been activated by FasL.

For example, one of the more tragic outcomes of runaway apoptosis in a specific organ is acute liver failure. Acute liver failure is characterized by an over-activation of the apoptotic pathway where there is massive apoptosis of hepatocytes and hemorrhagic liver changes. Acute liver failure can happen as a result of viral infections affecting the liver, bacterial infections affecting the liver, hepatitis, hepatocellular injury and/or other conditions where hepatocytes undergo massive apoptosis. One example of an infection resulting in acute liver failure is bacterium-induced fulminant hepatitis.

### SUMMARY OF THE INVENTION

The invention encompasses an isolated nucleic acid molecule having the sequence of FIG. 1, an isolated nucleic acid molecule having the sequence of FIG. 3, isolated polypeptide having the sequence of FIG. 1, and an isolated polypeptide having the sequence of FIG. 3. The invention also includes a transgenic mouse comprising a transgene having the sequence of FIG. 1.

The present invention encompasses a method for treating an individual suffering from abnormal hepatocyte apoptosis, comprising administration of a therapeutically amount of mFLINT protein to said individual. The invention also includes a method of treating an individual suffering from a disorder associated with inflammation, comprising administration of a therapeutically amount of mFLINT protein to said individual. Additionally, the invention includes a method of treating an individual suffering from abnormal apoptosis, comprising administration of a therapeutically effective amount of mFLINT protein to said individual.

The present invention further includes the use of mFLINT to treat a variety of disorders of the liver. In this regard, the invention encompasses a method of treating an individual suffering from acute liver failure, comprising administration of a therapeutically amount of mFLINT protein to said individual. The invention also includes a method of treating an individual suffering from inflammation of the liver, comprising administration of a therapeutically amount of mFLINT protein to said individual. Another method of the invention is for treating an individual suffering from hepatitis, comprising administration of a therapeutically effective amount of mFLINT protein to said individual.

The invention also encompasses a method of treating an individual suffering from sepsis, comprising administration of a therapeutically amount of mFLINT protein to said individual.

Also included within the present invention is a method for treating an individual suffering from an ischemia-associated injury or disorder, comprising administration of a therapeutically effective amount of mFLINT protein to said individual. Such an injury or disorder may associated with hypercoagulation. In this regard, the invention also includes treating a disorder associated with hypercoagulation, comprising administration of a therapeutically effective amount of mFLINT, in combination with an thrombolytic agent or an antithrombotic agent. One example of such an antithrombotic agent is activated protein C.

The invention also includes a method of treating an individual suffering from a reperfusion-associated injury or disorder, comprising administration of a therapeutically effective amount of mFLINT protein to said individual.

The invention further encompasses a method of preventing damage to a cardiac myocyte in an individual that has suffered from abnormal myocardial ischemia, comprising administration of a therapeutically effective amount of mFLINT protein to said individual.

Also included in the invention is a method of treating an individual suffering from Type I diabetes, comprising administration of a therapeutically amount of mFLINT protein to said individual. Yet another method encompassed by the present invention is a method of treating an individual suffering from cancer, comprising administration of a therapeutically effective amount of mFLINT protein to said individual.

The invention also includes a method of treating damage to an innocent bystander tissue that is induced by a chemotherapeutic agent or therapeutic irradiation, in an individual treated with said agent or irradiation, comprising administration of a therapeutically effective amount of mFLINT to said individual. Such tissues include, bone marrow and intestinal epithelium, including the epithelium of the oral cavity.

The invention encompasses method for treating hematopoietic progenitor cells that have been exposed to therapeutic radiation or chemotherapy, comprising administering mFLINT to said cells. Such a method promotes the recover of hematopoietic progenitor cells from the adverse effects of therapeutic radiation or chemotherapy. The invention also includes a method of promoting the growth or differentiation of a hematopoietic progenitor cell, comprising administering mFLINT to said cell. The invention further includes a method of promoting the growth or differentiation of a CD34+ cell, comprising administering mFLINT to said cell.

The invention also includes a method for treating cancer, comprising treating bone marrow cells in vitro with mFLINT, and administering said cells to said patient, wherein said administration occurs after said patient has been treated with therapeutic irradiation or chemotherapy. The bone marrow cells may be autologous, i.e., from the patient being treated, or heterologous, i.e., from an individual other the patient.

The invention also encompasses a method of treating cell damage in a patient who receives therapeutic irradiation or chemotherapy, comprising administering to said patient, a therapeutically effective amount of mFLINT with said irradiation or chemotherapy. The cell damage may be to an intestinal epithelial cell, a hematopoietic progenitor cell, or a peripheral blood cell.

The invention also contemplates methods of treating aplastic anemia, myelodysplastic syndrome or a pancytopenic condition, comprising administering a therapeutically effective amount of mFLINT to a patient suffering from aplastic anemia.

### BRIEF DESCRIPTION OF THE DRAWINGS

FIG. 1 shows the combined amino acid and nucleotide sequences of human FLINT.

FIG. 2 shows the combined amino acid and nucleotide sequences of human FLINT.

FIG. 3 shows the combined amino acid and nucleotide sequences of human mFLINT.

FIG. 4 shows the combined amino acid and nucleotide sequences of human mFLINT.

FIG. 5 compares the performance of mFLINT with other agents in an animal sepsis model after different times of LPS administration.

FIG. 6, left panel, shows an experiment comparing mFLINT with anti-TNF in an animal sepsis model. The right panel compares a lower dose of mFLINT against anti-TNF in the same model.

FIG. 7 shows an experiment comparing intravenous and intraperitoneal delivery of mFLINT and anti-TNF in an animal sepsis model.

FIG. 8 shows reduction in B16 melanoma tumor volume in response to treatment with mFLINT.

FIG. 9 shows mFLINT promoted recovery of bone marrow progenitor cells following irradiation.

FIG. 10 shows mFLINT promoted recovery of bone marrow progenitor cells following treatment with 5-fluorouracil.

### DETAILED DESCRIPTION OF THE INVENTION

By employing methods for identifying compounds that bind Fas ligand, applicants have discovered that human FLINT polypeptides are capable of disrupting FasL-Fas receptor interaction. Applicants have discovered methods for modulating the TNFR proteins and their respective ligand interactions where such interactions cause or exacerbate disease and methods for preventing or treating diseases.

As noted above, it is well established that one of the downstream effects of FasL-Fas receptor binding is apoptosis. In conditions evidencing abnormal activation of this pathway, runaway apoptosis results, which is a contributing factor in the pathology of a variety of disorders. Yet another downstream effect of FasL-Fas receptor binding is neutrophil activation wherein cells are destroyed by neutrophils activated by FasL.

Recently, it was discovered that FasL induces in peritoneal exudate cells (PEC) the processing and release of IL-1β that is responsible for the neutrophil infiltration. In particular, Miwa K., et al. Nature Medicine, 4(11): 1287-1292 (November 1998), found that inoculation of tumor cells expressing Fas ligand into wild-type mice induces a massive neutrophil infiltration that is, in contrast, suppressed in IL-1αβ knockout mice. This indicates that FasL has an inflammatory role. It also suggests that apoptosis may itself induce inflammation under certain conditions. In addition, it is known that certain inflammatory factors can induce the Fas-mediated apoptosis pathway. Thus, it is likely that FasL acts via two possibly distinct yet related pathways in exerting its pathological effects.

The inventors discovered that FLINT polypeptides bind to FasL with at least the same, if not greater, affinity than the Fas receptor itself. As a result of binding FasL, FLINT polypeptides can interfere with FasL binding to Fas receptor and interfere with events downstream. Using various in vitro and animal models, presented below in the examples, the inventors have demonstrated the ability of FLINT polypeptides to prevent Fas-mediated deleterious effects. It is apparent from these data that FLINT can act on both the apoptotic and proinflammatory aspects of FasL activity, which implicates its use in several disease and injury states discussed below.

Data presented below show that mFLINT inhibits both FasL apoptosis-inducing activity and proinflammatory activity. By antagonizing FasL, mFLINT polypeptides can modulate the destruction of healthy cells caused both by neutrophils activated by FasL and by apoptotic damage mediated directly by FasL-Fas interaction. Accordingly, the present methods of treatment utilizing mFLINT are useful in the treatment and prevention of disorders associated with the direct apoptotic effects of FasL and/or the damage mediated by the proinflammatory effects of FasL, whether or not these represent distinct physiological pathways.

Thus, as characterized generally, the invention relates to methods preventing or treating conditions caused or exacerbated by “abnormal apoptosis,” in particular, apoptosis induced by Fas ligand (FasL) and Fas receptor (Fas) binding (also referred to as FasL-Fas binding). This invention also relates to methods of preventing or treating conditions caused by a proinflammatory response, more particularly, a proinflammatory response caused by FasL induced neutrophil activation.

Throughout this application, headings are used for purposes of organizational convenience only, and should not be construed to limit the subject matter described herein.

I. Definitions

The following definitions are used in the present application.

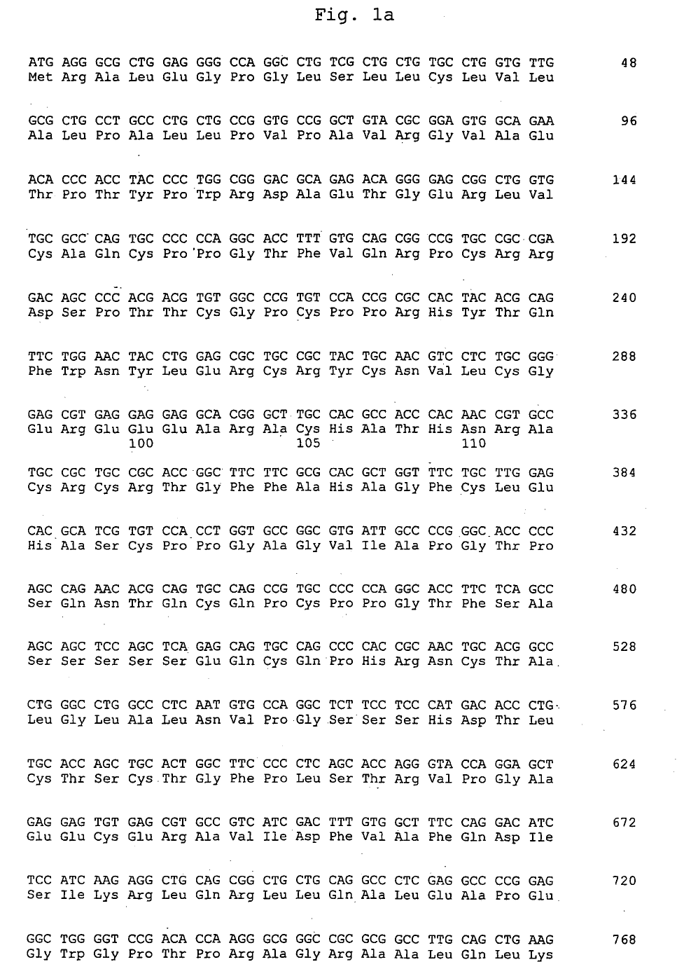

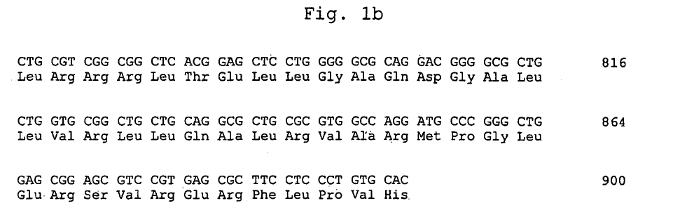

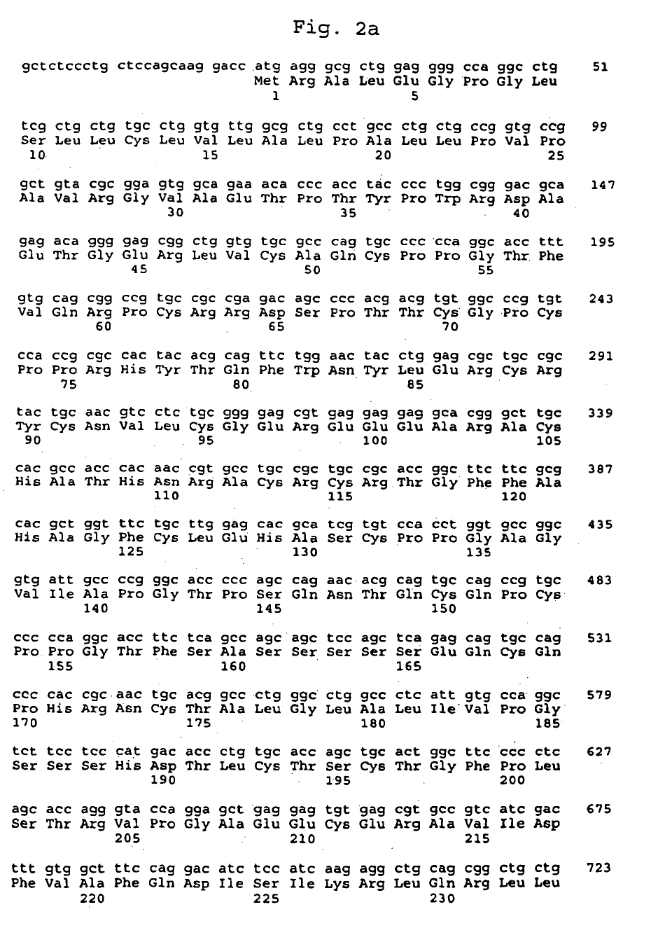

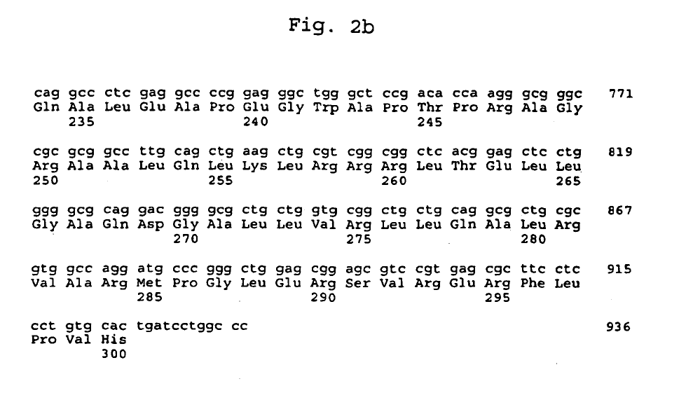

As used in this application, the term “FLINT” refers to any fill length FLINT polypeptide. Such a full-length polypeptide includes the leader sequence, which is amino acids 1-29 of a FLINT polypeptide. Examples of FLINT include a protein having the amino acid sequence set forth in FIG. 1, which is a human FLINT, and FIG. 2, which also is a human FLINT.

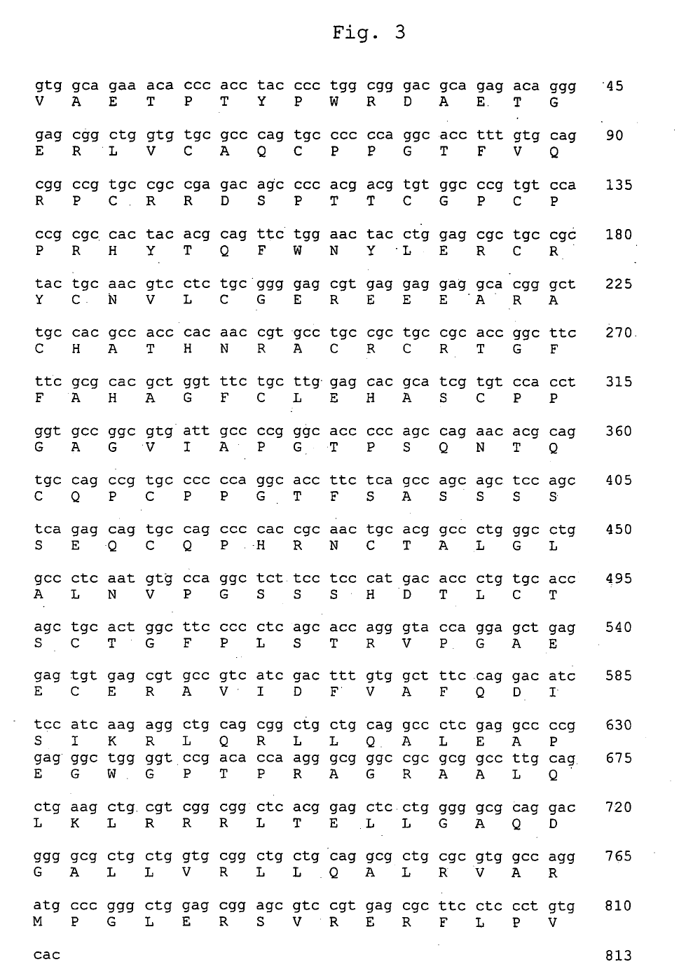

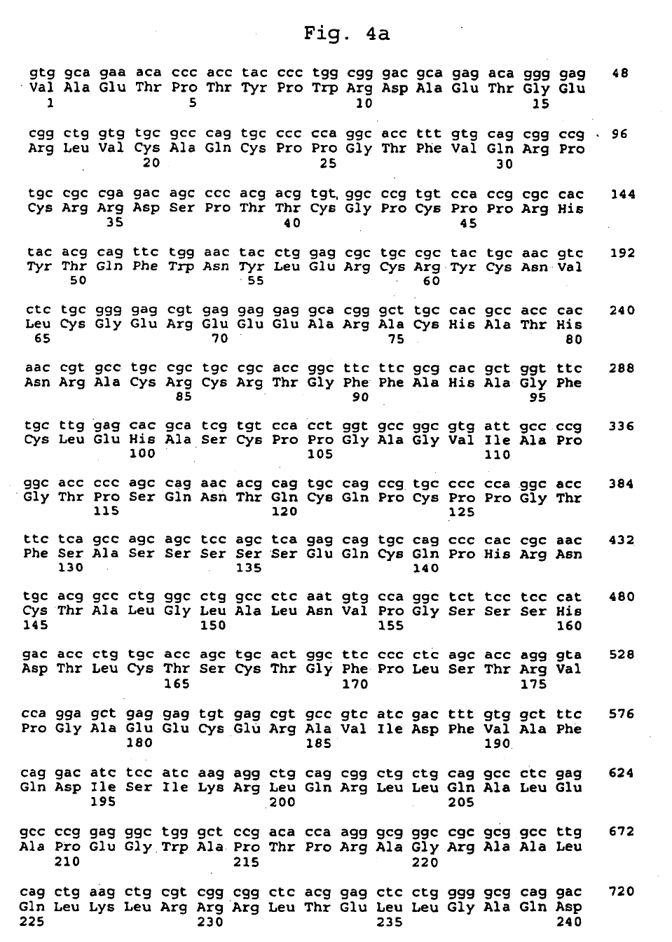

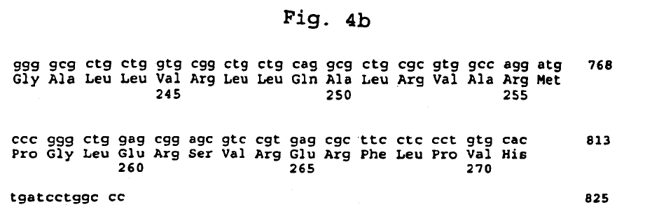

As used in this application, the term “mFLINT” refers to a mature FLINT, i.e., FLINT which does not have a leader (also known as signal) peptide. Examples of mFLINT include a protein having the amino acid sequence set forth in FIG. 3, which is the protein of FIG. 1 without its leader peptide, and a protein having the amino acid sequence set forth in FIG. 4, which is the protein of FIG. 2 without amino acids 1-29. Accordingly, an “mFLINT gene” is a nucleic acid that encodes an mFLINT polypeptide. The nucleic acids set forth in FIGS. 3 and 4 are examples of mFLINT genes according to the present invention.

As used here, with reference to FasL or Fas expression or interaction, and to any resulting apoptosis, the terms “inappropriate” and “abnormal” should be read to include any deviation from normal expression, interaction or apoptosis levels. Such deviations include temporal, quantitative and qualitative abnormalities. FasL or Fas “expression” refers not only transcription, translation and associated events, but also to any process that results in increased the availability of active FasL or Fas, such as transport and cell surface availability/accessibility.

Also as used herein the term “abnormal apoptosis” refers to excessive and/or improper apoptosis. Typically abnormal apoptosis is observed in cells and tissues that have undergone physical, chemical or biological insult. Such insults include, but are not limited to physical injury, viral infection, bacterial infection, ischemia, irradiation, chemotherapy, and the like.

The term “fusion protein” denotes a hybrid protein molecule not found in nature comprising a translational fusion or enzymatic fusion in which two or more different proteins or fragments thereof are covalently linked on a single polypeptide chain.

“Host cell” refers to any eucaryotic or prokaryotic cell that is suitable for propagating and/or expressing a-cloned gene contained on a vector that is introduced into said host cell by, for example, transformation or transfection, or the like:

“Isolated nucleic acid compound” refers to any RNA or DNA sequence, however constructed or synthesized, which is locationally distinct from its natural location.

The term “plasmid” refers to an extrachromosomal genetic element. The plasmids disclosed herein are commercially available, publicly available on an unrestricted basis, or can be constructed from readily available plasmids in accordance with published procedures.

A “primer” is a nucleic acid fragment which functions as an initiating substrate for enzymatic or synthetic elongation of, for example, a nucleic acid compound.

The term “promoter” refers to a nucleic acid sequence that directs transcription, for example, of DNA to RNA. An inducible promoter is one that is regulatable by environmental signals, such as carbon source, heat, or metal ions, for example. A constitutive promoter generally operates at a constant level and is not regulatable.

“Recombinant DNA cloning vector” as used herein refers to any autonomously replicating agent, including, but not limited to, plasmids and phages, comprising a DNA molecule to which one or more additional DNA segments can or have been incorporated.

The term “recombinant DNA expression vector” or “expression vector” as used herein refers to any recombinant DNA cloning vector, for example a plasmid or phage, in which a promoter and other regulatory elements are present thereby enabling transcription of an inserted DNA, which may encode a polypeptide.

The term “selectively binding” refers to the ability of FLINT polypeptides to bind FasL but not TNF∝.

“Substantially pure” used in reference to a peptide or protein means that said peptide or protein is separated from other cellular and non-cellular molecules, including other protein molecules. A substantially pure preparation would be about at least 85 % pure; preferably about at least 95 % pure. A “substantially pure” protein as described herein could be prepared by a variety of techniques well known to the skilled artisan, including, for example, the IMAC protein purification method.

The term “vector” as used herein refers to a nucleic acid compound used for introducing exogenous or endogenous DNA into host cells. A vector comprises a nucleotide sequence which may encode one or more protein molecules. Plasmids, cosmids, viruses, and bacteriophages, in the natural state or which have undergone recombinant engineering, are examples of commonly used vectors.

The various restriction enzymes disclosed and described herein are commercially available and the manner of use of said enzymes including reaction conditions, cofactors, and other requirements for activity are well known to one of ordinary skill in the art. Reaction conditions for particular enzymes were carried out according to the manufacturer's recommendation.

II. FLINT Binds Fas L and LIGHT

FLINT is a newly identified member of the TNFR superfamily. This family of receptors mediates a variety of biological effects of TNF ligands, including but not limited to cell proliferation, cell differentiation, immune regulation, inflammatory response, cytotoxicity, and apoptosis.

The FLINT polypeptide of the present invention is a soluble receptor comprising extracellular domains. FLINT polypeptide does not include any transmembrane domains and is, therefore, soluble. FLINT has also been called OPG3 (osteoprotegrin 3) or TNFRsol. FLINT is believed to be closely related to TNFR 6α and TNFR 6β discussed in WO98/30694 claiming priority to U.S.SNo. 60/035,496 and TR4, discussed in EP 0861850A1.

Data presented below demonstrate that mFLINT binds to FasL and to LIGHT in vitro. FasL, as noted, is involved in triggering both an inflammatory response and in inducing apoptosis. The biological activity for LIGHT includes, but is not limited to, cell proliferation. LIGHT is a 29 kDa type II transmembrane TNF superfamily member protein produced by activated T cells. Mauri D. M., Immunity, 8:21 (1998). As with FasL, evidence links LIGHT with neutrophil infiltration and with apoptosis. Zhai et al., J. Clin. Investig. 102:1142-51, 1998. Thus, mFLINT-mediated inhibition of LIGHT activity is expected to be therapeutically useful substantially as described below for mFLINT-mediated inhibition of FasL, especially in immune modulation and cancer therapy.

III. FLINT—Therapeutic Applications

The inventors have discovered that mFLINT prevents in vitro FasL-induced apoptosis of Jurkat cells. Anti-CD3 antibodies activate these cells and cause them to express FasL and undergo apoptosis. mFLINT also was effective in inhibiting in vitro anti-CD3-induced apoptosis of Jurkat cells, in a dose-dependent manner.

The present invention is applicable to the treatment and/or prevention of disorders associated with an inappropriate or abnormal interaction of FasL with Fas, because mFLINT prevents this interaction. This inappropriate interaction may arise, for example, due to increased expression or availability of FasL and/or Fas. Since it is known that FasL-Fas interaction induces apoptosis, the inventive methods further generally apply to disorders characterized by inappropriate or abnormal apoptosis.

In another embodiment the present invention relates to a method of preventing or treating conditions caused or exacerbated by FasL-Fas binding including FasL-mediated apoptosis and/or a proinflammatory response, more particularly, a proinflammatory response caused by FasL induced neutrophil activation.

For instance, mFLINT may be employed in treating Down's syndrome. Enhanced apoptosis of neuronal cells may be implicated in Down Syndrome. In this regard, Seidi, et al., Neuroscience Lett. 260:9 (1999), reported elevated levels of Fas protein in the temporal lobe and cerebellum of adult patients with Down syndrome, suggesting that Fas-associated apoptosis may be an important feature of neurodegeneration in Down syndrome. It is expected, therefore, that treatment of a Down syndrome patient with mFLINT may be effective. In particular, the present inventors have shown that mFLINT binds FasL, prevents Fas-FasL binding, and inhibits apoptosis. Similarly, apoptosis has been linked to Alzheimer's disease and other neurodegenerative diseases. Administration of mFLINT is expected to decrease the enhanced apoptosis associated with Down syndrome, Alzheimer's disease and other neurodegenerative disorders.

The inventors have tested mFLINT in a variety of model systems that are indicative of various disease and injury states. Some exemplary disorders include, but are not limited to, antibody-dependent cytotoxicity, infection with human immunodeficiency virus, hemolytic uremic syndrome, allergies and bronchopulmonary dysplasia. Thus, the following section discusses diseases and injury states in the context of corresponding model systems.

The present invention also encompasses the production of transgenic animals that contain a FLINT transgene. In particular, the inventors have produced transgenic mice that produce measurable levels of mFLINT. Animals such as these are useful for assessing the effects of mFLINT on a variety of disorders, detailed below, such as endotoxin-induced shock, cerebral ischemia, cardiac reperfusion injury, and damage induced by cancer therapies, such as therapeutic irradiation and chemotherapy.

A. Treatment of Liver Damage and Inflammation with mFLINT

FasL has been implicated in acute liver failure including but not limited to the damage caused to the liver in hepatitis. In a mouse model of acute hepatitis, administration of anti-FasL antibody to mice caused liver failure induced by apoptosis in hepatocytes and the animals die within hours. Kondo et al, 1997 Nature Medicine 3(4):409-413. See also Galle et al, J. Exp. Med, November 1995, 182:1223-1230.

Tsuji et al. (1998), Infect. Immun. 65:1892-1898, describes a model system for bacterially induced fluminant hepatitis. As detailed below, this system is predictive of a variety of disorders of the liver and other tissues. In the method of Tsuji et al., mice were injected with Propionibacterium acnes, and are subsequently injected with lipopolysaccharide (LPS). In normal mice, the first injection causes granuloma formation in the liver, while the second injection induces massive apoptosis and consequent liver damage. Tsuji et al. employed this method to implicate TNFRp55 in granuloma formation, and TNFRp55 and Fas in apoptosis.

Using a modified version of the animal model of Tsuji et al., the applicants have found that administration of mFLINT dramatically improves the survival rate of test animals. As indicated, many of the disorders treatable and/or preventable according to the present invention share a FasL-Fas-related etiology. Thus, target diseases generally involve either a tissue inappropriately (e.g., temporally, qualitatively or quantitatively) expressing Fas or inappropriately coming into contact with FasL while expressing Fas. Many of these diseases follow a general pathological model of inappropriate upregulation of Fas, followed by FasL-mediated induction of apoptosis. The inappropriate expression of Fas (or FasL) may be induced, for example, by inflammatory insult, with certain inflammation-associated cyokines likely inducing expression.

For instance, it is known that the first-phase mononuclear infiltration results in the secretion of numerous cytokines, which may result in inappropriate Fas and/or FasL expression. It is known, for example, that IL-1α, IL-1β and TNF-α can induce Fas expression. This increased Fas expression may, in effect, sensitize the affected tissue to FasL-bearing effector cells, like natural killer cells and cytotoxic T cells. Contact of the FasL-bearing effectors with Fas-bearing targets induces apoptosis of the latter. In other words this hepatic injury model is an in vivo surrogate for certain disease characterized by (a) inflammatory insult and/or (b) FasL-Fas-mediated apoptosis and/or necrosis.

Consistent with this model is the pathology of graft versus host disease (GVHD) which is very common, for example, in autologous bone marrow transplantation. GVHD results from the presence of host-reactive T cells that destroy host tissues, at least in part via FasL-mediated apoptosis. GVHD is typically divided into two phases, termed afferent and efferent. The afferent phase is characterized by recognition of host antigens and proliferation of donor T cells. In efferent phase tissues like the skin, liver and gastrointestinal tract become inflamed, and it is characterized by mononuclear cell infiltration and histopathological damage. Experiments using FasL-defective mice, in fact, show that FasL plays a key role in the development of hepatic, cutaneous and lymphoid organ damage in GVHD. Hence, GVHD follows the paradigm of inflammatory insult and Fas-mediated apoptosis.

Hashimoto's thyroiditis (HT) also fits this paradigm. HT results from an autoimmune response against the thyroid follicular cells. Normal thyrocytes produce FasL and express negligible levels of Fas. In HT, however; the resulting inflammation results in IL-1β secretion by activated macrophages, which then induces the thyrocytes to produce Fas. This sets up a fatal FasL-Fas autocrine loop that results in apoptosis.

Similarly, oxidized low density lipoprotein (OxLDL), associated with atherosclerotic lesions, promotes a chronic inflammatory response. While the vascular endothelium normally expresses both FasL and Fas, absent insult by OxLDL it does not undergo apoptosis. Upon treatment with OxLDL, however, FasL expression is increased and the endothelial cells undergo apoptosis.

Chronic renal failure is correlated secretion of IL-1α and TNF-α, both of which have been shown to induce Fas expression in renal tubular epithelial cells. This disorder is characterized by the depletion of tubular epithelial cells, via the FasL-Fas apoptosis pathway. Moreover, the same cytokine-mediated induction of Fas expression in tubule cells is observed in the endotoxic shock (sepsis) model of acute renal failure.

Acute liver failure can be found in viral infections affecting the liver, bacterial infections affecting the liver, hepatitis, hepatocellular injury and/or other conditions where hepatocytes undergo massive apoptosis. Galle et al, (J. Exp. Med., November 1995, 182:1223-1230), for example, found that FasL-Fas mediated cell death played a role in liver failure in humans. Galle et al. knew that FasL-Fas mediated apoptosis was a mechanism to eliminate senescent hepatocytes. For example, it was found in liver regeneration, during involution of liver after cessation of treatment with lipophyllic compounds (e.g., phenobarbitol) and during viral infection. Through experiments they found that during viral hepatitis, activated T cells attacked hepatocytes. Fas is constitutively expressed in hepatocytes. That is, FasL was expressed in T cells upon activation. It is thought that activated T cells might kill hepatitis B (HBV) antigen-expressing hepatocytes in an effort to clear HBV from the liver.

The inventors' success using a modified model of Tsuji further indicates that mFLINT is useful in treating and preventing sepsis. Sepsis is characterized by the presence of one or more pathogenic organisms, or their toxins, in the blood or tissues. Furthermore, sepsis is characterized by a systemic inflammatory response to infection that is associated with and mediated by the activation of a number of host defense mechanisms including the cytokine network, leukocytes, and the complement and coagulation/fibrinolysis systems. See Mesters, et al. Blood, 88:881 (1996).

It is known that endotoxin(s) induce tissue necrosis factor (TNF), which in turn induces both an inflammatory response and FasL, thereby promoting liver failure and death. Attempts to use antibodies drawn to TNF-α in treating sepsis, however, have uniformly failed. This is likely do to the fact that, aside from its effects on the pathology of sepsis, TNF-α is a more global mediator of normal immune function. In addition, as demonstrated below in the examples, TNF inhibitors show little usefulness in treating post-insult sepsis; rather they are more useful prophylactically.

Data presented below in the examples confirm that mFLINT is more effective than TNF inhibitors in promoting survival in an animal sepsis model. In fact, these data show that, whereas TNF inhibitors are efficacious only if used in a pre-treatment protocol (i.e., before LPS/galactosamine administration), mFLINT may be used in a post treatment regimen. This is clinically significant because the patient will virtually always present after endotoxic exposure. In other words, these data demonstrate that mFLINT is useful in both preventative and ameliorative regimens, in marked contrasted to TNF inhibitors. Minimally, these data indicate that mFLINT may be administered at a later time in disease progression than anti-TNF; it is thus expected to avoid the problems that were encountered with TNF inhibitors in the clinic.

Moreover, these data demonstrate that mFLINT has a therapeutic effect on at least two levels. Endotoxin in known to induce TNF, which directly induces both an inflammatory response and FasL. The data of Tsuji et al, supra, indicate that these represent two distinct pathways, both of which contribute to liver failure and death. In particular, their data using Fas-deficient mice strongly suggested that a substantial amount of the damage in their model is done through a Fas-independent, TNF-dependent pathway. The data presented below, however, demonstrate that mFLINT, an inhibitor of the Fas-dependent pathway, can inhibit all damage induced in the model, Fas-dependent or not. Thus, even if they represent different pathways, mFLINT can inhibit the damage mediated by both the inflammatory response and the induction of FasL.

In addition, it is contemplated that mFLINT may be beneficially used in combination with other agents useful in treating sepsis. For instance, U.S. Pat. No. 5,009,889 demonstrates that activated protein C (aPC) is effective in treating a baboon sepsis model. Such a model may be employed to determine appropriate combination treatment protocols using, for example, mFLINT and aPC. aPC may prevent the sepsis-associated disseminated intravascular coagulation and widespread deposition of fibrin in the microvasculature, which is an early manifestation of sepsis/septic shock.

Thus, based on the improved survival rate observed in mFLINT-treated animals, the inventors contemplate that one or more of the following diseases can be treated with mFLINT: acute respiratory distress syndrome (ARDS); goiter; heptatocellular injury (viral, bacterial, cancer, trauma); mononucleosis; mucocites (inflammation of the mucosa); pancreatitis; periodontal disease/gingivitis; renal failure; satellite organ failure; systemic inflammatory response syndrome (SIRS); surgery; vascular bleeds; vascular leak syndrome; whole organ transplant; multiple organ dysfunction (MODS); coronary artery bypass grafting; allograft rejection; transplant rejection; infection (e.g., microbial, pneumonia, tissue necrotic infection, viral); bone loss in rheumatoid arthritis; Hashimoto's thyroiditis; viruses, including hepatitis C (HCV), hepatitis B (HBV), Ebola (hemolytic fever) and Epstein-Barr (EBV); cutaneous inflammation; psoriasis; inflammatory bowel disease; ulcerative colitis; Crohn's disease; atherosclerosis; end stage renal disease; and sepsis.

More particularly, the invention contemplates methods for treating disorders associated with inflammation. The skilled artisan will recognize that these disorders include but are not limited to, GVHD, Hashimoto's thyroiditis, ARDS, heptatocellular injury (viral, bacterial, cancer, trauma); mucocites (inflammation of the mucosa); pancreatitis, periodontal disease/gingivitis; renal failure; satellite organ failure; systemic inflammatory response syndrome (SIRS); whole organ transplant; multiple organ dysfunction (MODS); coronary artery bypass grafting; allograft rejection; transplant rejection; infection (e.g., microbial, pneumonia, tissue necrotic infection, viral); rheumatoid arthritis; viral infections, including hepatitis C (HCV), hepatitis B (HBV), Ebola (hemolytic fever) and Epstein-Barr (EBV); cutaneous inflammation; inflammatory bowel disease; atherosclerosis; and sepsis.

B. Treatment of Ischemia and Reperfusion with mFLINT

Ischemia is characterized by the reduced blood flow to a tissue, which results in the accumulation of a variety of toxic metabolites. These metabolites contribute to cell death resulting from necrosis and/or apoptosis. Perhaps surprisingly, when a tissue is reperfused, apoptotic damage is increased. This is likely due to the generation of free radicals and other toxins through the reaction of ischemia-induced metabolites with serum components. In any event, studies have shown that the apopototic damage results at least in part from Fas-mediated pathways. Experiments presented below demonstrate that mFLINT can be used to block ischemia/reperfusion injury, likely by inhibiting FasL.

The usefulness of mFLINT compositions in treating or preventing disorders associated with cerebral ischemia, like stroke and head trauma, is confirmed by data presented below. There, a model is presented that uses a live gerbil model of global stroke. Cerebral ischemia was induced by a transient occlusion of the common carotid arteries followed by a treatment of mFLINT or a vehicle control. Data show that the mFLINT-treated gerbils retained markedly more living neurons than the vehicle treated control group.

These data are consistent with studies comparing the extent of infarct brain tissues in mice lacking functional Fas receptors after a cerebral ischemia insult, which found that significantly less damage than normal controls. Rosenbaum, et al., Annals of Neurology, 44(3),-441, 1998. It is, therefore, anticipated that mFLINT will interfere the normal signaling process of the Fas receptor in a patient that has undergone a cerebral ischemia episode thus protecting the brain tissues from deterioration.

Moreover, Fas-mediated apoptosis has been linked to ischemia reperfusion injury in a myocardial infarction model. Kajstura et al., Laboratory investigation 74:86-107 (1996). Thus, the foregoing gerbil model likely is predictive of generalized ischemia reperfusion injury. In support of this, the inventors conducted in an in vitro assay using cardiac myocytes. In particular, cardiomyocytes were incubated under hypoxia conditions for 8 hours, followed by 16 hours of culture under normal O₂, which mimics the hypoxic conditions found in ischemic cardiac tissue. Experimental results showed that treating the cardiomycetes with mFLINT protected the cells against apoptosis induced by hypoxia. In particular, treatment with 10 μg/ml mFLINT resulted in a 90% inhibition of hypoxia-induced apoptosis. FasL-induced apoptosis of cardiomycetes also was inhibited by treatment with mFLINT.

Based on the foregoing observations and data, the inventors contemplate that one or more of the following diseases can be treated with mFLINT: stroke; spinal cord ischemia; eclampsia/preeclampsia; reperfusion injury; myocardial infarction, its acute, subacute and chronic sequelae and related clinical syndromes, including but not limited to congestive heart failure. Thus, FLINT is useful in promoting myocardial salvage and preventing decompensatory myocardial hypertrophy.

Reperfusion injury may be in a number of tissues and result from a variety of insults, including the bowel, burns, cardiac bypass machine injury, hepatic (trauma-induced), hemolytic fever (Ebola), infant toxicity (hyperoxia incubators with high O₂ content), limb crush lung injury/ARDS, organ transplant, multiple trauma (e.g. from car accidents), protection of vascular beds, vascular organ failure usually present in sepsis and other diseases. These observations further indicate that mFLINT is useful in the prevention/attenuation of apoptosis of cardiac myocytes following acute myocarial infarction, and generally in preventing damage to cardiac and other tissue due to hypoxia.

In the case of organ preservation in preparation for harvesting, instance, mFLINT is useful prophylactically to prevent the apoptosis associated with ischemia reperfusion injury to the organ once it is removed from the donor. A typical method involves pretreating the donor with an effective amount of mFLINT prior to organ harvesting. Alternatively, or conjunctively, the harvested organ may be perfused or bathed in a mFLINT-containing solution. This method may be employed, for example, with kidney, heart, lung and other organs and tissues.

In another example, mFLINT is useful in treating the ischemia reperfusion injury associated with thrombus formation or hypercoagulation. Thus, mFLINT may be administered in-conjunction with thrombolytic agents (e.g., urokinase and streptokinase) and/or antithrombotics, like activated protein C (aPC). U.S. Pat. No. 5,350,578 describes the antithrombotic activity of aPC. The baboon animal model disclosed therein may be used to ascertain a beneficial dosing regimen for the contemplated combination therapy. In this regard, it is expected that the following disorders may be treated with mFLINT, with or without the addition of aPC: eclampsia and preeclampsia, which are characterized by a state of increased coagulopathy; HELLP (preeclampsia complicated by thrombocytopenia, hemolysis, and disturbed liver function), HITS (heparin-induced thrombocytopenia); disseminated intravascular coagulation (DIC); burns; and thromboembolic complications that result from surgery.

As noted above, studies show that a great deal of apoptotic injury is induced upon reperfusion. This injury appears to be due at least in part to oxidative damage, since antioxidants can diminish the injury. Likewise, it has been found that Vitamin E, an antioxidant, can be used as a meat preservative, perhaps acting to prevent the same type of injury, which causes spoilage. Administering Vitamin E to pigs several days before slaughter has been shown to increase shelf-life of pork. Hence, it is contemplated that mFLINT is similarly useful to preserve tissues and organs. This would be particularly useful for increasing shelf-life of organs and meats for human consumption.

C. Treatment of Hematopoietic Disorders with mFLINT

As discussed in this application, mFLINT inhibits Fas/FasL interaction, thereby preventing apoptosis. As discussed below, the Fas/FasL interaction has been implicated in the process of hematopoiesis. Therefore, the present application contemplates mFLINT-based therapeutic methods for improving hematological recovery from a variety of treatments and disorders, including bone marrow transplantation, chemotherapy, radiotherapy, aplastic anemia, and myelodysplastic syndrome, pancytopenic conditions. The present invention also covers the mFLINT-based treatment of any clinical condition which requires expansion of bone marrow cell lineages, alone or in combination with other hematological growth factors. Such growth factors include, but are not limited to, erythropoietin (EPO), FLT-3 ligand, thrombopoietin (TPO), stem cell factor (SCF), granulocyte colony stimulating factor (G-CSF), and granulocyte-macrophage colony-stimulating factor (GM-CSF).

Autologous and heterologous bone marrow transplantation are commonly used to treat a multitude of neoplastic diseases. In autologous transplantation, bone marrow cells are removed from the patient to be treated, and are cultured in vitro. Following chemotherapy and/or radiation therapy, the cells are re-introduced to the patient. In heterologous transplantation, the bone marrow cells are donated by a second individual. The chemotherapy or radiotherapy that is used to treat such diseases results in a dramatic myelosuppression from damage to the bone marrow compartments and intestinal epithelium, leaving the patient immunosuppressed and susceptible to invasion by microorganisms. The treatment of transplanted bone marrow cells with hematopoietic cytokines has been shown to result in improved recovery of the myeloid and erythroid lineages of the blood, although there typically is toxicity associated with the use of these cytokines and one cytokine typically does not enhance recovery of all lineages. Moore, M. A. S. Blood 78:1 (1991); Metcalf, D. Science 254: 529 (1991).

Radiation or chemotherapy has been shown to induce apoptosis of bone marrow cells and intestinal epithelial cells. The Fas/FasL pathway is known to induce apoptosis in susceptible cells. Recent reports describe the involvement of Fas/FasL in hematopoiesis. For a review, see Niho, et al.; Current Opinion in Hematology, 5: 163 (1998). Fas is expressed on CD34+ progenitor cells and these cells are susceptible to the apoptotic effects of Fas stimulation. Nagafuji et al., Blood, 86: 883-889 (1995), Sato et al., Br J Haematol, 97:. 356 (1997). Yamane et al., Eur J Haematol, 58:289 (1997). Moreover, in vitro expansion of hematopoietic progenitor cells with cytokines has been shown to upregulate functional Fas expression and it is currently thought that Fas may play an in vivo role in hematopoietic homeostasis as a negative regulator. Takenaka et al., Blood, 88: 2871 (1996), Stahnke et al., Exp. Hematol. 26: 844 (1998).

1. mFLINT and Radiation and Chemotherapy

The inventors have shown that mFLINT improves the recovery of bone marrow progenitor cells that have been exposed to irradiation. In particular, mice were irradiated, and bone marrow cells were removed and cultured in vitro with the cytokines IL-6 and CSF. The addition of mFLINT to the culture medium dramatically increased the recovery of bone marrow progenitor cells, namely progenitors of erythroid cells, granulocyte-macrophage cells, and erythroid monocyte megakaryocyte cells. The inventors also have shown that mFLINT improves the survival of bone marrow cells that were harvested from mice treated with 5-fluorouracil (5-FU). Anti-FasL antibody also increased the survival of the cells taken from 5-FU-treated mice.

The present invention thus encompasses the use of mFLINT as a radioprotective and/or chemoprotective agent. mFLINT is useful for enhancing the in vitro expansion and maturation of bone marrow progenitor cells prior to autologous or heterologous transplantation. mFLINT also is useful for promoting the expansion and maturation of progenitor cells, after a patient has been treated with radiation or chemotherapy. In this regard, administration of mFLINT to a patient is expected to promote the expansion and maturation of progenitor cells when that patient has been treated with a cancer therapy (chemo- or radio-therapy). mFLINT also is expected to promote the expansion and maturation of progenitor cells in patients treated with cancer therapy, and myelosuppressive agent, which prevents cycling of progenitor cells during cancer therapy.

As noted above, cytokines are used, in vitro and in vivo, to enhance the expansion of erythroid and myeloid cell lineages in patients receiving cancer therapies. The present inventors have shown that mFLINT improves the expansion of cells that are treated with cytokines. Accordingly, the present invention contemplates the use of mFLINT, in combination with one or more cytokines, to expand bone marrow progenitor cells, in vitro and in vivo. Granulocyte-macrophage colony stimulating factor (GM-CSF) is used to improve recovery from myelosuppression that results from chemo- and radio-therapy. Accordingly, the present invention contemplates the use of a combination of mFLINT and GM-CSF to improve recovery from myelosuppression.

The inventors also have shown that anti-FasL antibody also improved the recovery of bone marrow cells from treatment with 5-FU. Accordingly, the present invention contemplates the use of anti-FasL to improve recovery of bone marrow cells, following chemo- or radio-therapy. Such an antibody can be used in vitro to expand bone marrow cells for heterologous or autologous transplantation. An anti-FasL antibody also can be administered in vivo to improve recovery from myelosuppression that results from chemotherapy, radiation therapy, and/or administration of myelosuppressive agents.

Systemic administration would be appropriate when-treating patients with mFLINT. As used in this application, administration of mFLINT “with” chemotherapy or irradiation encompasses all of the following modes of administration of mFLINT: (1) pretreatment with mFLINT, followed by irradiation or chemotherapy; (2) simultaneous administration of mFLINT and chemo- or radiation therapy; (3) chemotherapy or irradiation first, followed by mFLINT administration; (4) mFLINT pretreatment, followed by chemo- or radiation therapy, followed by administration with mFLINT. The invention encompasses the use of mFLINT in one or more of the foregoing modes of treatment.

2. Treatment of Peripheral Cytopenias

The present invention also contemplates the treatment of peripheral cytopenias that are associated with hematopoietic disorders, such as aplastic anemia and myelodysplastic syndrome. Fas/FasL-mediated apoptosis has been shown to suppress lymphopoiesis. Yasutomo, et al. J. Immunol. 157: 1981 (1996). Increased Fas expression is observed in progenitor cells from patients with aplastic anemia. Young, N. S., Eur. J. Hematol. 60 (supp):. 55(1996). Increased Fas expression also has been observed in progenitor cells from patients suffering from myelodysplastic syndrome.

Furthermore, Maria, et al. reported that Fas is rapidly upregulated in early erythroblasts and is expressed at high levels thorough terminal maturation. Blood 93:796 (1999). Maria, et al. also reported that immature blood cells are EPO-dependent and express a functional Fas molecule. In the presence of FasL-producing mature erythroblasts, the immature cells undergo apoptosis, unless exposed to high levels of EPO. Maria, et al. suggest that Fas/FasL system, in combination with EPO, contribute to erythropoietic homeostasis.

While not intending the bound to any particular theory, the reduction in hematopoiesis and resultant cytopenias may be a direct result of apoptosis, which may in turn be mediated through upregulation of Fas and Fas ligand. As described elsewhere in this application, mFLINT inhibits Fas/FasL binding, inhibiting apoptosis, and mFLINT also enhanced the growth and maturation of hematopoietic progenitor cells. Therefore, the use of mFLINT is expected to suppress apoptosis suffering from hematopoietic disorders, leading to amelioration of one or more of the symptoms associated with these disorders. Moreover, because immature hematopoietic cells are susceptible to FasL-induced apoptosis, and because individuals suffering from a lack of differentiated hematopoietic cells, the administration of mFLINT to such individuals is expected to ameliorate these maturation defects, by inhibiting FasL-induced apoptosis of immature hematopoietic cells.

Therapy of individuals suffering from hematopoietic disorders can be carried out by direct administration of mFLINT to the affected individual, or by the use of mFLINT in vitro to treat bone marrow cells that are then transplanted into the diseased individual.

Therapy with mFLINT can be augmented by administration of EPO and other compounds that promote the growth and/or maturation of hematopoietic progenitor cells. Cytokines are known to stimulate the growth of hematopoietic progenitor cells. Accordingly, the invention also encompasses the use of a combination of mFLINT and one or more cytokines to promote growth and differentiation of such progenitor cells in individuals suffering from diseases such as thrombocytopenia and myelodysplastic syndrome.

3. Gene Therapy

FLINT also can be used in conjunction with gene therapy. When hematopoietic progenitor cells are to be transplanted into an individual, they are transfected with a suitable transgene, and also are treated with mFLINT, optionally in combination with one or more cytokines. Following culturing of the cells, they are transplanted into an individual. Such gene therapy will be useful in imparting desirable properties to the blood cells.

D. Protection of Innocent Bystander Tissues with mFLINT

It is well known that many cell-damaging therapies, such as chemotherapy and therapeutic irradiation used to treat cancer, cause damage and apoptosis of so-called “innocent bystander” tissues. As used in this application, an “innocent bystander” tissue is a non-diseased tissue that is damaged by pharmacological, radiation or device-assisted therapies. These tissues include, but are not limited to, the gastric epithelium (including the epithelium lining the oral cavity, esophagus, stomach and intestinal tract), the lung epithelium, blood cells (lymphocytes, monocytes, T cells, B cells, bone marrow cells, hematopoietic progenitor cells, neutrophils, eosinophils, mast cells, platelets), renal epithelium, and hair follicles. Many of these tissues are characterized by rapid growth and/or turnover of their constituent cells.

“Cell damaging” therapies include, but are not limited to chemotherapy (e.g. cisplatin, doxorubicin, mitomycin C, camptothecin, and fluorouracil and other nucleoside analogs), therapeutic irradiation, laser treatment, and administration of inhaled toxins (such as bleomycin). Physical manipulations that are associated with device-assisted therapies, such as the physical removal of a blockage from a coronary artery, also can cause damage to innocent bystander tissue. While these cell-damaging therapies are useful because they damage and kill diseased tissue, as noted above, they have the undesirable side effect of damaging and inducing apoptosis of “innocent bystander” tissue.

As discussed elsewhere in this application, mFLINT blocks Fas/FasL interactions. It also has been demonstrated herein that mFLINT inhibits FasL-induced apoptosis in vitro, and that mFLINT improved the survival of bone marrow cells following therapeutic irradiation and chemotherapy. Therefore, it is an object of the present invention to ameliorate damage to innocent bystander tissues that is caused by cell-damaging therapy.

Cancer therapies such as irradiation and chemotherapy have been shown to induce apoptosis in intestinal epithelial cells. It is known that many chemotherapeutic agents work by inducing apoptosis. See, e.g., Micheau, et al. J. Natl. Can. Inst. 89:783 (1997). This apoptosis, combined with the myelosuppression that is induced by irradiation and chemotherapy, permits massive opportunistic infection. There currently are no effective therapies for managing the disruption of intestinal function that is associated with chemotherapy and therapeutic irradiation. As noted above, treatment with cytokines has been shown to improve recovery from myelosuppression, but cytokines can produce undesirable toxicity.

Therefore, it is expected that mFLINT will ameliorate damage to the intestinal epithelium that is induced by irradiation and/or chemotherapy. While not desiring to be bound to any particular theory, it is expected that mFLINT will ameliorate this damage by inhibition of apoptosis and/or inflammation of the intestinal epithelium.

Fas/FasL interactions have been implicated in chronic gastritis. It has been shown that Fas and FasL expression are increased in gastric epithelial cells from individuals suffering from chronic gastritis. See Rudi, et al., J. Clin. Invest. 102:1506 (1998). Rudi also reported that Helicobacter-infected gastric epithelial cells have increased levels of FasL (CD95 ligand) and Fas (CD95 receptor), and that Helicobacter-induced apoptosis was reduced by blocking FasL with anti-APO-1 antibody. Thus, it is expected that mFLINT may be effective in inhibiting (i) gastritis that is induced by Helicobacter and, as mentioned above, (ii) cell-damaging therapies, such as chemotherapy and irradiation.

Furthermore, patients receiving high-dose chemotherapy (HD-CT) are at risk of severe mucositis, which is characterized by apoptosis and inflammation of the gastric epithelium. Wymenga, et al., Br. J. Cancer 76:1062 (1997) studied mucositis of in the oral cavity of breast cancer patients treated with HD-CT. The percentage of viable epithelial cells appearing in an oral wash increased in patients treated with HD-CT, suggested a desquamation of the upper oral mucosal layer. Also, there was a higher percentage of immature cells in the oral mucosa of the HD-CT patients.

Other innocent bystander tissues also may benefit from mFLINT therapy. For example, Fas/FasL-induced apoptosis has been implicated in bleomycin-induced apoptosis and fibrosis in lung epithelium. Hagimnoto, et al. Am. J. Respir. Cell. Mol. Biol. 16:91 (1997). That study showed that in alveolar epithelial cells treated with bleomycin, Fas mRNA was upregulated, and FasL mRNA was upregulated in infiltrating lymphocytes. Therefore, the applicants expect that mFLINT therapy may ameliorate damage in lung tissue, such as epithelium, that is induced by cell-damaging therapies.

E. Treatment of Cancer with mFLINT

The present inventors have found that administration of mFLINT to mice causes a reduction in the volume of tumors that are produced when mice are injected with melanoma cells. This effect of mFLINT may be mediated through mFLINT/FasL binding and the consequent inhibition of FasL-mediated T-cell apoptosis, permitting a T-cell mediated immune response to facilitate destruction of the tumor. In this regard, it is known that FasL is expressed in melanomas, human colorectal cancers, hepatocellular cancers, astrocytomas and lung cancer. O'Connell, et al. Immunology Today insert*, Chappell, et al. Cancer Immunol. Immunother. 47:65 (1998). It also has been reported that colon cancer cell lines express FasL and can kill T cells in vitro by inducing FasL-mediated apoptosis. O'Connell. Therefore, the melanoma tumors observed in the present application may express FasL, triggering FasL-mediated death of infiltrating T-cells that express Fas. However, other mechanisms may also be responsible for the tumor reduction that has been demonstrated in the present application.

Based on the success achieved by the present inventors with mFLINT-mediated reduction of tumor volume, it is expected that other types of cancer also can be successfully treated with mFLINT. These cancers include, but are not limited to, astrocytoma, colon cancer, esophageal cancer, lung cancer, melanoma and hepatocellular cancer. Other cancers that can be treated with mFLINT include bladder cancer and ovarian cancer.

F. Treatment of Autoimmune Disease with mFLINT

Both Type I (insulin-dependent) and Type II (non-insulin dependent) diabetes are characterized by hyperglycemia. As used herein, the term hyperglycemia,” which is well known in the art, describes a condition characterized by a blood glucose level that is higher than that found in a normal human. Normal human fasting blood glucose levels are less than 110 mg/dL. The Expert Committee on the Diagnosis and Classification of Diabetes Mellitus, Diabetes Care, 21 (Suppl. 1), S5-S19 (1992).

Type I insulin-dependent diabetes (IDDM) is a chronic autoimmune disorder involving destruction of pancreatic (insulin-producing) β cells via the FasL-Fas pathway. It is likely that this destructive pathway is activated by the inflammatory process. Normal β cells do not express Fas, but Fas is expressed in these cells during insulitis. It is thus likely that lymphocyte-associated FasL contributes to the local destruction of these Fas⁺ cells via a FasL-Fas interaction. To the extent that mFLINT is useful in antagonizing this pathway, it is useful in preventing or treating IDDM.

Multiple sclerosis (MS) is a degenerative inflammatory demyelinating disease. Fas has be found among the oligodendrocytes located along the lesion margin and in the adjacent white matter in both acute and chronic MS, whereas normal tissue shows little-or none. It is likely that this abnormal expression of Fas is induced by cytokines released during the inflammatory process. Moreover, FasL-expressing cells have been co-localized with Fas-expressing apoptotic oligodendrocytes, suggesting involvement of this pathway in the pathology of this disease.

G. Treatment of Osteoporosis with mFLINT

Data presented below in the examples indicate that mFLINT is useful in preventing or treating disorders of bone loss, like osteoporosis. These data are consistent with data showing the naturally-occurring secreted member of the TNFR superfamily was recently reported as having a role in regulating bone resorption. Simonet et al. Cell 89:309(1997) (termed osteoprotegrin (OPG); Tsuda et al. Biochem., and Biophys. Res. Comm. 234:137 (1997) (termed osteoclastogenesis inhibitory factor (OCIF))), functioning essentially as inhibitors of differentiation of bone-resorbing osteoclasts. Consistently, studies have directly linked Fas to osteoblast apoptosis. Jilka et al., 1998, J. Bone & Mineral Res. 13:793-802; Kawakami et al., 1997, J. Bone & Mineral Res. 12:1637-46.

IV. Therapeutic Formulations of mFLINT

The mFLINT polypeptide composition will be formulated and dosed in a fashion consistent with good medical practice, ting into account the clinical condition of the individual patient (especially the side effects of treatment with mFLINT polypeptide alone), the site of delivery of the mFLINT polypeptide composition, the method of administration, the scheduling of administration, and other factors known to practitioners.

An effective amount of polypeptide results in a statistically significant modulation of the biological activity of the selected TNFR family ligand, for example, FasL or LIGHT. The biological activity for FasL includes, but is not limited to, apoptosis. The biological activity for LIGHT includes, but is not limited to, cell proliferation. LIGHT is a 29 kDa type II transmembrane TNF superfamily member protein produced by activated T cells. Mauri d.M., Immunity, 8:21-30, January 1998.

Further, an effective amount may also be determined by prevention or amelioration of adverse conditions or symptoms of diseases, injuries or disorders being treated. The “therapeutically effective amount” of mFLINT polypeptide for purposes herein is thus determined by such considerations. It should be noted that mFLINT is an immunomodulator and that a common observation with such substances is a bell-shaped dose-response curve. Such a phenomenon is well known in the art and it is within the skill of the clinician to take this into account in adjusting the therapeutically effective amount of mFLINT accordingly.

As a general proposition, the total pharmaceutically effective amount of mFLINT polypeptide administered parenterally per dose will be in the range of about 1 μg/kg/day to 10 mg/kg/day of patient body weight, more particularly 2-8 mg/kg, preferably 2-4 mg/kg, most preferred 2.2 mg/kg to 3.3 mg/kg and finally 2.5 mg/kg. However, as noted above, this will be subject to therapeutic discretion. Preferably, this dose is at least 0.01 mg/kg/day.

If given continuously, the mFLINT polypeptide is typically administered at a dose rate of about 0.1 μg/kg/hour to about 50 μg/kg/hour, either by 1-4 injections per day or by continuous subcutaneous infusions, for example, using a mini pump. An intravenous bag solution may also be employed. The length of treatment needed to observe changes and the interval following treatment for responses to occur appears to vary depending on the desired effect.

Pharmaceutical compositions containing the mFLINT of the invention may be administered using a variety of modes that include, but are not limited to, oral, rectal, intra-cranial, parenteral, intracisternal, intravaginal, intraperitoneal, topical, transdermal (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include but are not limited to, intravenous, intramuscular, intraperitoneal, intrasternal, subcutaneous and intraarticular injection and infusion. Implants comprising mFLINT also can be used.

The mFLINT polypeptide is also suitably administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773.919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988); Sustained-release “mFLINT polypeptide compositions also include liposomally entrapped mFLINT polypeptides. Liposomes containing mFLINT polypeptides are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EDP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30-mol. percent cholesterol, the selected proportion being adjusted for the optimal TNFR polypeptide therapy.

For parenteral administration, in one embodiment, the mFLINT polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the mFLINT polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The mFLINT polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of mFLINT polypeptide salts.

FLINT polypeptides to be used for therapeutic administration must be sterile. Sterility is readily accomplished by filtration through sterile filtration membranes (e.g., 0.2 micron membranes). Therapeutic mFLINT polypeptide compositions generally are placed into a container having a sterile access port, for example, an intravenous solution bag or vial having a stopper pierceable by a hypodermic injection needle.

FLINT polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous mFLINT polypeptide solution, and the resulting mixture is lyophilized. The infusion solution is prepared by reconstituting the lyophilized mFLINT polypeptide using bacteriostatic Water-for-Injection.

The invention also provides a pharmaceutical pack or kit comprising one or more containers filled with one or more of the ingredients of the pharmaceutical compositions-of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

V. Polypeptide Production Methods

One embodiment of the present invention relates to the substantially purified polypeptide encoded by a mFLINT gene or a mFLINT gene.

Skilled artisans will recognize that the polypeptides of the present invention can be synthesized by a number of different methods, such as chemical methods well known in the art, including solid phase peptide synthesis or recombinant methods. Both methods are described in U.S. Pat. No. 4,617,149.

The principles of solid phase chemical synthesis of polypeptides are well known in the art and may be found in general texts in the area. For example, see H. Dugas and C. Penney, BIOORGANIC CHEMISTRY (1981) Springer-Verlag, New York, 54-92. For example, peptides may be synthesized by solid-phase methodology utilizing an Applied Biosystems 430A peptide synthesizer (Applied Biosystems, Foster City, Calif.) and synthesis cycles supplied by Applied Biosystems.

The polypeptides of the present invention can also be produced by recombinant DNA methods using the cloned FLINT gene. Recombinant methods are preferred if a high yield is desired. Expression of the cloned gene can be carried out in a variety of suitable host cells, well known to those skilled in the art. For this purpose, the FLINT gene is introduced into a host cell by any suitable means, well known to those skilled in the art. While chromosomal integration of the cloned gene is within the scope of the present invention, it is preferred that the gene be cloned into a suitable extra-chromosomally maintained expression vector so that the coding region of the FLINT gene is operably-linked to a constitutive or inducible promoter.

The basic steps in the recombinant production of the FLINT polypeptide are:

a) constructing a natural, synthetic or semi-synthetic DNA encoding FLINT polypeptide;

b) integrating said DNA into an expression vector in a manner suitable for expressing the FLINT polypeptide, either alone or as a fusion polypeptide;

c) transforming or otherwise introducing said vector into an appropriate eukaryotic or prokaryotic host cell forming a recombinant host cell;

d) culturing said recombinant host cell in a manner to express the FLINT polypeptide; and

e) recovering and substantially purifying the FLINT polypeptide by any suitable means well known to those skilled in the art.

1. Expressing Recombinant FLINT Polypeptide in Prokaryotic and Eukaryotic Host Cells

Prokaryotes may be employed in the production of recombinant FLINT polypeptide. For example, the Escherichia coli K12 strain 294 (ATCC No. 31446) is particularly useful for the prokaryotic expression of foreign polypeptides. Other strains of E. coli, bacilli such as Bacillus subtilis, enterobacteriaceae such as Salmonella typhimurium or Serratia marcescans, various Pseudomonas species and other bacteria, such as Streptomyces, may also be employed as host cells in the cloning and expression of the recombinant polypeptides of this invention.

Promoter sequences suitable for driving the expression of genes in prokaryotes include β-lactamase [e.g. vector pGX2907, ATCC 39344, contains a replicon and β-lactamase gene], lactose systems [Chang et al., Nature (London), 275:615 (1978); Goeddel et al., Nature (London), 281:544 (1979)], alkaline phosphatase, and the tryptophan (trp) promoter system [vector pATH1 (ATCC 37695)], which is designed to facilitate expression of an open reading frame as a trpE fusion polypeptide under the control of the trp promoter. Hybrid promoters such as the tac promoter (isolatable from plasmid pDR540, ATCC-37282) are also suitable. Still other bacterial promoters, whose nucleotide sequences are generally known, may be ligated to DNA encoding the polypeptide of the instant invention, using linkers or adapters to supply any required restriction sites. Promoters for use in bacterial systems also will contain a Shine-Dalgarno sequence operably-linked to the DNA encoding the desired polypeptides. These examples are illustrative rather than limiting.

The polypeptides of this invention may be synthesized either by direct expression or as a fusion polypeptide comprising the polypeptide of interest as a translational fusion with another polypeptide or peptide that may be removed by enzymatic or chemical cleavage. It often is observed in the production of certain peptides in recombinant systems that expression as a fusion polypeptide prolongs the lifespan, increases the yield of the desired peptide, or provides a convenient means of purifying the polypeptide. This is particularly relevant when expressing mammalian polypeptides in prokaryotic hosts. A variety of peptidases (e.g. enterokinase and thrombin) which cleave a polypeptide at specific sites or digest the peptides from the amino or carboxy termini (e.g. diaminopeptidase) of the peptide chain are known. Furthermore, particular chemicals (e.g. cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites. For instance, see P. Carter, “Site Specific Proteolysis of Fusion Polypeptides”, Chapter 13, in PROTEIN PURIFICATION: FROM MOLECULAR MECHANISMS TO LARGE SCALE PROCESSES, American Chemical Society, Washington, D.C. (1990).

In addition to prokaryotes, a variety of amphibian expression systems such as frog oocytes, and mammalian cell systems can be used. The choice of a particular host cell depends to some extent on the particular expression vector used. Exemplary mammalian host cells suitable for use in the present invention include HepG-2 (ATCC HB 8065), CV-1 (ATCC CCL 70), LC-MK₂ (ATCC CCL 7.1), 3T3 (ATCC CCL 92), CHO-K1 (ATCC CCL 61), HeLa (ATCC CCL 2), RPMI8226 (ATCC CCL 155), H4IIEC3 (ATCC CCL 1600), C127I (ATCC CCL 1616), HS-Sultan (ATCC CCL 1484), and BHK-21 (ATCC CCL 10), for example.

A wide variety of vectors are suitable for transforming mammalian host cells. For example, the pSV2-type vectors comprise segments of the simian virus 40 (SV40) genome required for transcription and polyadenylation. A large number of plasmid pSV2-type vectors have been constructed, such as pSV2-gpt, pSV2-neo, pSV2-dhfr, pSV2-hyg, and pSV2-β-globin, in which the SV40 promoter drives transcription of an inserted gene. These vectors are widely available from sources such as the American Type Culture Collection (ATCC), 12301 Parklawn Drive, Rockville, Md., 20852, or the National Center for Agricultural Utilization Research, 1815 North University Street, Peoria, Ill. 61604-39999.

Promoters suitable for expression in mammalian cells include the SV40 late promoter, promoters from eukaryotic genes, such as, for example, the estrogen-inducible chicken ovalbumin gene, the interferon genes, the glucocorticoid-inducible tyrosine aminotransferase gene, the thymidine kinase gene promoter, and the promoters of the major early and late adenovirus genes.

Plasmid pRSV cat (ATCC 37152) comprises portions of a long terminal repeat of the Rous Sarcoma virus, a virus known to infect chickens and other host cells. This long terminal repeat contains a promoter which is suitable for use in the vectors of this invention. H. Gorman et al., Proc. Nat. Acad. Sci. (USA), 79,-6777 (1982). The plasmid pMSVi (NRRL B-15929) comprises the long terminal repeats of the Murine Sarcoma virus, a virus-known to infect mouse and other host cells. The mouse metallothionein promoter has also been well characterized for use in eukaryotic host cells and is suitable for use in the present invention. This promoter is present in the plasmid pdBPV-MMTneo (ATCC 37224) which can serve as the starting material for the construction of other plasmids of the present invention.,

Transfection of mammalian cells with vectors can be performed by a plurality of well known processes including, but not limited to, protoplast fusion, calcium phosphate co-precipitation, electroporation and the like. See, e.g., Maniatis et al., supra.

Some viruses also make appropriate vectors. Examples include the adenoviruses, the adeno-associated viruses, the vaccinia virus, the herpes viruses, the baculoviruses, and the rous sarcoma virus, as described in U.S. Pat. No. 4,775,624, incorporated herein by reference. For example, the baculovirus pFastBac-1 (GIBCO/BRL) can be used to infect a suitable host cell, such as SF9, to produce recombinant protein.

Eukaryotic microorganisms such as yeast and other fungi are also suitable host cells. The yeast Saccharomyces cerevisiae is the preferred eukaryotic microorganism. Other yeasts such as Kluyveromyces lactis and Pichia pastoris are also suitable. For expression in Saccharomyces, the plasmid YRp7 (ATCC-40053), for example, may be used. See, e.g., L. Stinchcomb et al., Nature, 282, 39 (1979); J. Kingsman et al., Gene, 7, 141 (1979); S. Tschemper et al., Gene, 10, 157 (1980). Plasmid YRp7 contains the TRP1 gene which provides a selectable marker for use in a trp1 auxotrophic mutant.

Purification of Recombinantly-Produced FLINT Polypeptide

An expression vector carrying the cloned FLINT gene is transformed or transfected into a suitable host cell using standard methods. Cells that contain the vector are propagated under conditions suitable for expression of the recombinant FLINT polypeptide. For example, if the recombinant gene has been placed under the control of an inducible promoter, suitable growth conditions would incorporate the appropriate inducer. The recombinantly-produced polypeptide may be purified from cellular extracts of transformed cells by any suitable means.

In a preferred process for polypeptide purification, the FLINT gene is modified at the 5′ end to incorporate several histidine residues at the amino terminus of the FLINT polypeptide. This “histidine tag” enables a single-step polypeptide purification method referred to as “immobilized metal ion affinity chromatography” (IMAC), essentially as described in U.S. Pat. No. 4,569,794, which hereby is incorporated by reference. The IMAC method enables rapid isolation of substantially pure recombinant FLINT polypeptide starting from a crude extract of cells that express a modified recombinant polypeptide, as described above.

Other embodiments of the present invention comprise isolated nucleic acids that encode FIGS. 1, 2,3,4. Any of these nucleic acids may be produced by chemical synthetic methods. The synthesis of nucleic acids is well known in the art. See, e.g., E. L. Brown, R. Belagaje, M. J. Ryan, and H. G. Khorana, Methods in Enzymology, 68:109-151 (1979). Fragments of the DNA sequence corresponding to a FLINT or an “mFLINT gene could be-generated using a conventional DNA synthesizing apparatus, such as the Model 380A or 380B DNA synthesizers of Applied Biosystems, Inc. (850 Lincoln Center Drive, Foster City, Calif. 94404), using phosphoramidite chemistry, and thereafter ligating the fragments so as to reconstitute the entire gene. Alternatively, phosphotriester chemistry may be employed to synthesize nucleic acids used in this invention. See, for example, Gait, M. J., ed. OLIGONUCLEOTIDE SYNTHESIS, A PRACTICAL APPROACH (1984).

In an alternative methodology, namely PCR, the DNA sequences disclosed and described herein, comprising, for example, FIG. 1 can be produced from a plurality of starting materials. For example, starting with a cDNA preparation (e.g. cDNA library) derived from tissue that expresses the FLINT gene, suitable oligonucleotide primers complementary to FIG. 1 or to any sub-region therein, are prepared as described in U.S. Pat. No. 4,889,818. Other suitable protocols for the PCR are disclosed in PCR PROTOCOLS: A GUIDE TO METHOD AND APPLICATIONS, Ed. Michael A. Innis et al., Academic Press, Inc. (1990).

The ribonucleic acids of the present invention may be prepared via polynucleotide synthetic methodology, discussed above, or they may be prepared enzymatically, for example, by using RNA polymerase to transcribe a FLINT DNA template. The most preferred systems for preparing the ribonucleic acids of the present invention employ the RNA polymerase from the bacteriophage T7 or the bacteriophage SP6. These RNA polymerases are highly specific, requiring the insertion of bacteriophage-specific sequences at the 5′ end of the template to be transcribed. See Maniatis et al., supra. This invention also provides nucleic acids, RNA or DNA, that are complementary to FIGS. 14.

2. Vectors

Another aspect of the present invention relates to recombinant DNA cloning vectors and expression vectors comprising the nucleic acids of the present invention.

The skilled artisan understands that choosing the most appropriate cloning vector or expression vector depends upon a number of factors including the availability of restriction enzyme sites, the type of host cell into which the vector is to be transfected or transformed, the purpose of the transfection or transformation (e.g., stable transformation as an extrachromosomal element, or integration into the host chromosome), the presence or absence of readily assayable or selectable markers (e.g., antibiotic resistance and metabolic markers of one type and another), and the number of copies of the gene desired in the host cell.

Vectors suitable to carry the nucleic acids of the present invention comprise RNA viruses, DNA viruses, lytic bacteriophages, lysogenic bacteriophages, stable bacteriophages, plasmids, viroids, and the like. The most preferred vectors are plasmids.

When preparing an expression vector the skilled artisan understands that there are many variables to be considered, for example, whether to use a constitutive or inducible promoter. The practitioner also understands that the amount of nucleic acid or polypeptide to be produced dictates, in part, the selection of the expression system. Regarding promoter sequences, inducible promoters are preferred because they enable high level, regulatable expression of an operably-linked gene. The skilled artisan will recognize a number of suitable promoters that respond to a variety of inducers, for example, carbon source, metal ions, and heat. Other relevant considerations regarding an expression vector include whether to include sequences for directing the localization of a recombinant polypeptide. For example, a sequence encoding a signal peptide preceding the coding region of a gene is useful for directing the extra-cellular export of a resulting polypeptide.

The present invention also provides a method for constructing a recombinant host cell capable of expressing polypeptides comprising FIGS. 1-4. This method comprises transforming or otherwise introducing into a host cell a recombinant DNA vector that comprises an isolated DNA sequence as described in any of FIGS. 1 through 4.

The preferred host cell is any eukaryotic cell that can accommodate high level expression of an exogenously introduced gene or polypeptide, and that will incorporate said polypeptide into its membrane structure. Vectors for expression are those which comprise any of the sequences of FIGS. 1-4. Transformed host cells, may be cultured under conditions well known to skilled artisans such that FLINT or mFLINT is expressed, thereby producing a recombinant FLINT or mFLINT polypeptide in the recombinant host cell.

The following examples more fully describe the present invention. Those skilled in the art will recognize that the particular reagents, equipment, and procedures described are merely illustrative and are not intended to limit the present invention in any manner.

### EXAMPLE 1

### RT-PCR Amplification of FLINT Gene from mRNA

A FLINT gene is isolated by reverse transcriptase PCR (RT-PCR) using conventional methods. Total RNA from a tissue that expresses the FLINT gene, for example, lung, is prepared using standard methods. First strand FLINT cDNA synthesis is achieved using a commercially available kit (SuperScript™ System; Life Technologies) by PCR in conjunction with specific primers directed at any suitable region of FIGS. 1-4.

Amplification is carried out by adding to the first strand cDNA (dried under vacuum): 8 μl of 10X synthesis buffer (200 mM Tris-HCl, pH 8.4; 500 mM KCl, 25 mM MgCl₂, 1 ug/ul BSA); 68 μl distilled water; 1 μl each of a 10 uM solution of each primer; and 1 μl Taq DNA polymerase (2 to 5 U/μl). The reaction is heated at 94° C. for 5 minutes to denature the RNA/cDNA hybrid. Then, 15 to 30 cycles of PCR amplification are performed using any suitable thermal cycle apparatus. The amplified sample may be analyzed by agarose gel electrophoresis to check for an appropriately-sized fragment.

### EXAMPLE 2

### Production of a Vector for Expressing FLINT in a Host Cell

An expression vector suitable for expressing FLINT or fragment thereof in a variety of prokaryotic host cells, such as E. coli is easily made. The vector contains an origin of replication (Ori), an ampicillin resistance gene (Amp) useful for selecting cells which have incorporated the vector following a transformation procedure, and further comprises the T7 promoter and T7 terminator sequences in operable linkage to a FLINT coding region. Plasmid pET11A (obtained from Novogen, Madison Wis.) is a suitable parent plasmid. pET11A is linearized by restriction with endonucleases NdeI and BamHI Linearized pET11A is ligated to a DNA fragment bearing NdeI and BamHI sticky ends and comprising the coding region of the FLINT gene as disclosed by FIGS. 1-4.

The FLINT gene used in this construction may be slightly modified at the 5′ end (amino terminus of encoded polypeptide) in order to simplify purification of the encoded polypeptide product. For this purpose, an oligonucleotide encoding 8 histidine residues is inserted after the ATG start codon. Placement of the histidine residues at the amino terminus of the encoded polypeptide serves to enable the IMAC one-step polypeptide purification procedure.

### EXAMPLE 3

### Recombinant Expression and Purification of FLINT Polypeptide

An expression vector that carries an open reading frame (ORF) encoding FLINT or fragment thereof and which ORF is operably-linked to an expression promoter is transformed into E. coli BL21 (DE3)(hsdS gal λcIts857 ind1Sam7nin5lacUV5-T7gene 1) using standard methods. Transformants, selected for resistance to ampicillin, are chosen at random and tested for the presence of the vector by agarose gel electrophoresis using quick plasmid preparations. Colonies which contain the vector are grown in L broth and the polypeptide product encoded by the vector-borne ORF is purified by immobilized metal ion affinity chromatography (IMAC), essentially as described in U.S. Pat. No. 4,569,794.

Briefly, the IMAC column is prepared as follows. A metal-free chelating resin (e.g., Sepharose 6B IDA, Pharmacia) is washed in distilled water to remove preservative substances and infused with a suitable metal ion [e.g., Ni(II), Co(II), or Cu(II)] by adding a 50 mM metal chloride or metal sulfate aqueous solution until about 75% of the interstitial spaces of the resin are saturated with colored metal ion. The column is then ready to receive a crude cellular extract containing the recombinant polypeptide-product.

After removing unbound polypeptides and other materials by washing the column with any suitable buffer, pH 7.5, the bound polypeptide is eluted in any suitable buffer at pH 4.3, or preferably with an imdizole-containing buffer at pH 7.5.

### EXAMPLE 4

### Tissue Distribution of FLINT mRNA

The presence of FLINT mRNA in a variety of human tissues was analyzed by Northern analysis. Total RNA from different tissues or cultured cells was isolated by a standard guanidine chloride/phenol extraction method, and poly-A⁺ RNA was isolated using oligo(dT)-cellulose type 7 (Pharmacia). Electrophoresis of RNA samples was carried out in formaldehyde followed by capillary transfer to Zeta-Probe™ nylon membranes (Bio-Rad, Hercules, Calif.). FIG. 1 was the template for generating probes using a MultiPrime™ random priming kit (Amersham, Arlington Heights, Ill.). The efficiency of the labeling reaction was approximately 4×10¹⁰ cpm incorporated per μg of template. The hybridization buffer contained 0.5M sodium phosphate, 7% SDS (wt/vol), 1% BSA (wt/vol), and 1 mM EDTA. Prehybridizati6n was carried out in hybridization buffer at 65° C. for 2 h and ³²P-labeled probe was added and incubation continued overnight. The filters were washed in Buffer A (40 mM sodium phosphate pH 7.2, 5% SDS [wt/vol], 0.5% BSA [wt/vol], and 1 mM EDTA) at 65° C. for 1 h, and then in Buffer B (40 mM sodium phosphate, pH 7.2, 1% SDS [wt/vol], and 1 mM EDTA) at 65° C. for 20 minutes. The filters were air-dried and exposed to Kodak X-OMAT AR film at −80° C. with an intensifying screen.

The results showed that FLINT mRNA was present in numerous tissues, including stomach, spinal cord, lymph node, trachea, spleen, colon and lung.

### EXAMPLE 5

### Production of an Antibody to a Polypeptide

Substantially pure polypeptide or fragment thereof is isolated from transfected or transformed cells using any of the well known methods in the art, or by a method specifically disclosed herein. Concentration of polypeptide in a final preparation is adjusted, for example, by filtration through an Amicon filter device such that the level is about 1 to 5 ug/ml. Monoclonal or polyclonal antibody can be prepared as follows.

Monoclonal antibody can be prepared from murine hybridomas according to the method of Kohler and Milstein (Nature, 256, 495, 1975), or a modified method thereof. Briefly, a mouse is repetitively inoculated with a few micrograms of the polypeptide or fragment thereof, or fusion peptide thereof, over a period of a few weeks. The mouse is then sacrificed and the antibody producing cells of the spleen isolated. The spleen cells are fused by means of polyethylene glycol with mouse myeloma cells. Fused cells that produce antibody are identified by any suitable immunoassay, for example, ELISA, as described in E. Engvall, Meth. Enzymol. 70, 419, 1980.

Polyclonal antiserum can be prepared by well known methods, as described, for example, by J. Vaitukaitis et.al., Clin. Endocinol. Metab. 33, 988 (1971), that involve immunizing suitable animals with the polypeptides, fragments thereof, or fusion polypeptides thereof, disclosed herein. Small doses (e.g., nanogram amounts) of antigen administered at multiple intradermal sites appears to be the most reliable method.

### EXAMPLE 6

### Construction of Mammalian FLINT-Flag Expression Vector

To facilitate confirmation of FLINT expression (without the use of antibodies), a bicistronic expression vector (pIG1-FLINTF) was constructed by insertion of an “internal ribosome entry site”/enhanced green fluorescent polypeptide (IRES/eGFP) PCR fragment into the mammalian expression vector pGTD (Gerlitz, B. et al., 1993, Biochemical Journal 295:131). This new vector, designated pIG1, contains the following sequence landmarks: the E1a-responsive GBMT promoter (D. T. Berg et al., 1993 BioTechniques 14:972; D. T. Berg et al., 1992 Nucleic Acids Research 20:5485); a unique BclI cDNA cloning site; the IRES sequence from encephalomyocarditis virus (EMCV); the eGFP (Clontech) coding sequence (Cormack, et al., 1996 Gene 173:33); the SV40 small “t” antigen splice site/poly-adenylation sequences; the SV40 early promoter and origin of replication; the murine dihydrofolate reductase (dhfr) coding sequence; and the pBR322 ampicillin resistance marker/origin of replication. The resultant protein had the Flag sequence attached at the C-terminal end, yielding: [FLINT]-DYKDDDDK.

Based upon the human FLINT sequence, the following primers were synthesized: 5′-TAGGGCTGATCAAGGATGGGCTTCTGGACTTGGGCGGCCCC TCCGCAGGCGGACCGGGG-3′; and 5′-AGGGGGGCGGCCGCTGATCATCACTT GTCGTCGTCGTCCTTGTAGTCGTGCA CAGGGAGGAAGCGC-3′. The latter, reverse primer contains the Flag epitope sequence (positions 24-47, double underline) (Micele, R. M. et al., 1994 J. Immunol. Methods 167:279). These primers were then used to PCR amplify the FLINT cDNA. The resultant 1.3 Kb PCR product was then digested with BclI (restriction sites incorporated into primers, underlined above) and ligated into the unique BclI site of pIG1 to generate the plasmid pIG1-FLINTF. The human FLINT cDNA orientation and nucleotide sequence were confirmed by restriction digest and double stranded sequencing of the insert.

### EXAMPLE7

Construction of Mammalian FLINT-non-Flag Expression Vector

In order to generate a non-Flagged expression vector (pIG1-FLINT), the 24-base DNA sequence encoding the eight amino acid FLAG epitope was deleted from the pIG1-FLINTF construct using the Quick Change mutagenesis kit (Stratagene). A 35-base primer, and its complement, with identity to the 19-base sequences flanking the FLAG sequence was synthesized and used for PCR amplification using pIG1-FLINTF the plasmid as template. The PCR reaction mixture was digested with DpnI restriction endonuclease to eliminate the parental DNA, and the PCR product was transformed into Epicurean XLI-blue E. coli cells. Sixteen ampicillin-resistant transformants were picked and the plasmid DNA was analyzed by restriction digestion. Ten of the 16 gave results compatible with deletion of the 24-base sequence. Precise deletion of the 24-base sequence was confirmed by DNA sequencing of pIG1-FLINT. The nucleotide sequence of FLINT in this plasmid is shown in FIG. 1.

### EXAMPLE 8

### Isolation of a High-Producing FLINT Clone from AV12 RGT18 Transfectants

The recombinant plasmid carrying the FLINT gene (pIG1-FLINT) encodes resistance to methotrexate. In addition, the construct contains a gene encoding a fluorescent polypeptide, GFP, on the same transcript and immediately 3′ to the FLINT gene. Since high level expression of GFP would require a high level of expression of the FLINT-GFP mRNA, highly fluorescent clones would have a greater probability of producing high levels of FLINT. pIG1-FLINT and pIG1-FLINTF were used to transfect AV12 RGT18 cells. Cells resistant to 250 nM methotrexate were selected and pooled. The pool of resistant clones was subjected to fluorescence assisted cell sorting (FACS), and cells having fluorescence values in the top 5% of the population were sorted into a pool and as single cells. The high fluorescence pools were subjected to three successive sorting cycles. Pools and individual clones from the second and third cycles were analyzed for FLINT production by SDS-PAGE. Pools or clones expressing FLINT at the highest level judged from Coomassie staining were used for scale-up and FLINT purification. The amino acid sequence of the mature FLINT protein that was produced using this plasmid is shown in FIG. 3, called mFLINT.

### EXAMPLE 9

### Large Scale mFLINT Polypeptide Purification

Large scale production of mFLINT was carried out by first growing stable clones stable pIG1-:FLINT-containing AV12 RGT 18 cells in several 10 liter spinners. After reaching confluency, cells were further incubated for 2-3 more days to secrete maximum amount of FLINT into media. Media containing mFLINT was adjusted to 0.1 % CHAPS and concentrated in an Amicon ProFlux M12 tangential filtration system to 350 ml. The concentrated media was centrifuged at 19,000 rpm (43,000×g) for 15 minutes and passed over a SP-5PW TSK-GEL column (21.5 mm×15 cm; TosoHaas) at a flow rate of 8 ml/min. The column was washed with buffer A(20 mM MOPS, 0.1 % CHAPS, pH 6.5) until the absorbency (280 mm) returned to baseline and the bound polypeptides were eluted with a linear gradient from 0.1 M-0.3 M NaCl(in buffer A) developed over 85 min. Fractions containing mFLINT were pooled and passed over a (7.5 mm×7.5 cm) Heparin-5PW TSK-GEL column equilibrated in buffer B (50 mM Tris, 0.1% CHAPS, 0.3 M NaCl, pH 7.0). The bound polypeptide was eluted with a linear gradient from 0.3 M-1.0 M NaCl (in buffer B) developed over 60 min. Fractions containing mFLINT were pooled and passed over a 1 cm×15 cm Vydac C4 column equilibrated with 0.1% TFA/H₂O. The bound mFLINT was eluted with a linear gradient from 0-100% CH₃CN/0.1% TFA. Fractions containing mFLINT were analyzed by SDS-PAGE and found to be greater than 95% pure and were dialyzed against 8 mM NaP0₄, 0.5 M NaCl, 10% glycerol,. pH 7.4. The N-terminal sequence of mFLINT was confirmed on the purified polypeptide. Mass spectral analysis and Endogylcosidase-F digestion indicates that mFLINT is glycosylated.

### EXAMPLE 10

A. Construction of FLINT-Flag and FLINT Expression Vectors Flag-tagged and native versions of human FLINT were expressed in baculovirus-infected cells in order to generate enough recombinant protein for study. The human FLINT cDNA was engineered for expression as follows. A pIG3 (a derivative of pIG1; Gerlitz, B and Grinnell, B. W., unpublished data) expression vector containing the cDNA encoding a FLAG-tagged version of FLINT was cleaved with XbaI and filled in to create blunt ends. The vector was then cleaved with SalI. The resulting 918-base pair fragment containing the coding region was gel-purified and ligated into the baculovirus vector pFastBac-1 (GIBCO/BRL) that had been digested with BamHI, the ends blunted and subsequently digested with Sail generating the plasmid pBacOPG3Flag. This construct was designed to express a full-length molecule (including the 29 NH2-terminal amino acids, which constitute the signal peptide) and the FLAG tag at the COOH-terminus of the protein. The expression was under the control of the baculovirus polyhedrin promoter.

For the construction of a vector to express the native version of the protein, a 920-base pair XbaI/HindIII cDNA fragment encoding the full-length of the protein previously subcloned into pCDNA3.1 (±) [purchased from Invitrogen] was cleaved with XbaI and HindIII. The fragment was gel-purified and ligated into the baculovirus vector pFastBac-1 (GIBCO/BRL) that had been digested with XbaI and HindIII generating the plasmid pBacOPG3.

B. Generation of Baculoviruses and Protein Production The two vectors, pBacOPG3Flag and pBacOPG3 were separately used to generate two recombinant baculoviruses (vBacOPG3Flag and vBacOPG3) as described by the vendor (GIBCO/BRL). Each of the viruses was separately used to infect SF-9 cells for protein production. The recombinant proteins were measured in supernatants collected from the infected SF-9 cells by Western-blot and Coomassie stain analyses.

### EXAMPLE11

### Fas Ligand Binding Experiments

To Detect mFLINT Interaction with FasL

Dot blot experiment was performed to scan known TNF ligands that are commercially available TRAIL and FasL for interaction with mFLINT.

TRAIL (RnD Systems) and FasL (Kamiya Biomedical Company) were spotted on a nitrocellulose paper and incubated with purified mFLINT-Flag. mFLINT was washed away and binding mFLINT was detected using anti Flag antibody. Both OPG2Fc and mFLINT-Flag were overexpressed and purified according the examples above. The filter paper was subsequently blocked for 30 min using 5% nonfat milk in PBS in room temperature. The nitrocellulose paper was subsequently mixed with the cell lysate containing FasL-Myc, and further incubated on a rotator for 1 hour at room temperature. Secondary and tertiary incubations were performed with anti-myc antibody and anti-mouse IgG-HRP for 1 hour and 30 minutes respectively. The polypeptide containing myc epitope was detected by chemiluminescence on X-ray film which showed that mFLINT bound to FasL specifically. No appreciable binding was detected with TNFα, TNFβ, TRAIL, CD40 L or TRANCE.

First a baseline experiment was done for the Fas-FasLigand interaction in vitro. Unless otherwise indicated, all washing steps use TBST (Tris Buffer Saline with Tween 20 from SIGMA) and were done 3 to 6 times.

mrecFas (100 ng) was adsorbed on to ELISA plate. Then the plate was is blocked by TBST plus 0.1% Gelatine. Thereafter, hFasLigand (Flag-tagged) was added at different concentrations with a maximum concentration of 300 ng going down to 1 ng on TBST plus a 0.1% solution containing 1 micrograms/ml of M2 Abs (antiflag antibodies purchased through Scientific Imaging System division of Kodak). After washing the plate 6 times, anti-mouse-Abs-HRP (3000 dilution, Bio-Rad) was added to the wells. After washings three times, visualization enzymatic reaction using ABTS as a substrate was performed. Unless otherwise noted, an ELISA reader commercialized by Molecular Devices Corp. (Menlo Park, Calif.) was used.

The following data were collected:

| FasL, ng | OD, 405 nM |
|---|---|
| 1 | .1 |
| 5 | .2 |
| 10 | .3 |
| 50 | .7 |
| 100 | 1.2 |
| 500 | 1.6 |

FLINT-Fas L binding may be confirmed and specifics of binding determined (e.g., kinetics, specificity, affinity, cooperativity, relative binding pattern, concentration) using real-time biomolecular interaction analysis. This technology confers the ability to study biomolecular interactions in real time, without labeling any of the interactants. In particular, it takes advantage of the optical phenomenon surface plasmon resonance, and detection depends on changes in the mass concentration of macromolecules at the biospecific interface. Interactions are followed in real time, so that kinetic information is readily derived. In many cases, investigations can be performed without prior purification of components.

Measurements are accomplished using a BiaCore 2000 instrument. The instrument, accompanying chips, immobilization and maintenance kits and buffers are obtained from Biacore AB, Rapsgatan 7, S-754 50 Uppsala, Sweden. FasL is obtained from Kamiya Biomedical Company, 910 Industry Drive, Seattle, Wash. 98188, Guanidine Isothiocyanate Solution from GibcoBRL, and mFLINT is prepared as in Examples 8 and 9.

Experiments using immobilized FasL loaded with a solution containing mFLINT. In this experiment, the K_{D} of the FasL-FLINT interaction was 1.13×10⁻⁷, which is lower than the FasL binding Fas, which was found to be 1.62×10⁻⁷. This is explained by the fact that FasL is a trimer, yet here FasL monomer was bound. Further experiments utilized bound FLINT with FasL in solution, allowing for trimer formation. The resultant K_{D} for FLINT-FasL in these experiments was 2 orders of magnitude better (i.e., the K_{D} was lower by two orders of magnitude). This observations indicate that FLINT should effectively compete with Fas for FasL binding, which likely explains the therapeutic benefit observed in experiments detailed below.

FLINT Prevents Fas-FasLigand Interaction

As above, mrecFas (100 ng) was adsorbed on to ELISA plate. Again the plate was blocked by TBST and 0.1% Gelatine. Thereafter, hFasLigand (Flag-tagged, 30 ng per each point) in the presence of different mFLINT concentrations (Maximum concentration 300 ng down to 1 ng) on TBST plus a 0.1% solution containing 1 microgram/ml of M2 Abs is added to each well. As before, after washing of the plate, anti-mouse-Abs-HRP (3000 dilution, Bio-Rad) was added to the wells. After washings, visualization enzymatic reaction using ABTS as a substrate was performed. The data is shown in the following table.

| FLINT, ng | OD, 405 nM |
|---|---|
| 1 | 0.36 |
| 5 | 0.36 |
| 10 | 0.36 |
| 50 | 0.28 |
| 100 | 0.18 |
| 500 | 0.06 |

FasLigand Binds Fas and mFLINT with Different Affinities.

FLINT and Fas (100 ng of each) were adsorbed on to an ELISA plate. hFasLigand (Flag-tagged) was added at different concentrations to a maximum concentration of 300 ng down to 0.1 ng on TBST plus a 0.1% solution containing 1 microgram/ml of M2 Abs. After washing of the plate, anti-mouse-Abs-HRP (1:3000 dilution, Bio-Rad) was added to the wells. After washings, visualization enzymatic reaction using ABTS as a substrate was performed. The table below shows the data.

| FasL, ng | FLINT OD | Fas OD, 405 nM |
|---|---|---|
| 0.1 | 0 | 0 |
| 0.5 | 0 | 0 |
| 1.0 | .02 | 0 |
| 5.0 | .04 | .01 |
| 10 | .12 | .03 |
| 50 | .28 | .045 |
| 100 | .78 | .18 |

### EXAMPLE 12

### Measuring the Effect of mFLINT on anti-CD3 Induced Jurkat Apoptosis

Non-tissue treated 24 well plates (Decton Dickinson, Mansfield, Mass.) were coated with 0.5 ml of 1 ug/ml anti-CD3 (Farmingen) in PBS for 90 min at 37° C. The plate was washed once with PBS. 1 ml of 1×10⁶ cell/ml was seated in each well with or without following treatment: 10 μM DEVD-cmk, 1 ug OPG2-Fc, 1 or 2 ug of mFLINT and 1 ug anti FasL Ab. mFLINT was made according to Examples 8 and 9.

Cells were incubated overnight at 37° C. incubator and cells were then stained by Annexin V and PI staining. Apotosis was analyzed by flow cytometer (FACS). Cell apoptosis was indicated by positive staining with Annexin V.

| | |
|---|---|
| Control Jurkat | 6.97 |
| Jurkat + anti Fas | 59.28 |
| Jurkat + antiCD3 | 46.32 |
| Jurkat + antiCD3 + DEVDcmk | 30.80 |
| Jurkat + antiCD3 + mFLINT (1 ug) | 27.77 |
| Jurkat + antiCD3 + OPG2-Fc (1 ug) | 45.78 |
| Jurkat + antiCD3 + mFLINT (2 ug) | 18.67 |
| Jurkat + antiCD3 + antiFasL Ab | 24.05 |

### EXAMPLE 13

### Measuring the Effect of mFLINT on Recombinant FasL Induced Jurkat Cells Apoptosis

One milliliter of 1×10⁶ cell/ml was added into each well of 24 well tissue culture plate and treated with following reagents: soluble Fas L (200 ng), Fas L plus 1 ug mFLINT, Fas L plus 1 ug OPG2-Fc, Trail (200 ng), Trail plus 1 ug mFLINT. Cells were incubated overnight at 37° C. and then stained with Annexin V and PI. Cell apoptosis was analyzed by flow cytometer (FACS). mFLINT was made according to Examples 8 and 9.

| | |
|---|---|
| Control Jurkat | 3.23 |
| Jurkat + FasL (200 ng/ml) | 67.39 |
| Jurkat + FasL (200 ng/ml) + | 3.3 |
| anti FasL Ab (1 ug) | |
| Jurkat + FasL (200 ng/ml) + mFLINT (1 ug) | 3.32 |
| Jurkat + FasL (200 ng/ml) + mFLINT (1 ug) | 4.6 |
| Jurkat + FasL (200 ng/ml) + OPG2 (1 ug) | 70.58 |
| Jurkat + FasL (200 ng/ml) + OPG2 (1 ug) | 69.58 |
| Jurkat + TRAIL (200 ng/ml) | 17.47 |
| Jurkat + TRAIL (200 ng/ml) | 17.43 |

### EXAMPLE 14

### Measuring the Effect of mFLINT in a Dose-Dependent Manner on Anti-CD3 Induced Jurkat Apoptosis

The same steps for plate coating and cell treatment set out in Example 13 were followed except a different amount of mFLINT was added into each well. mFLINT was made according to Examples 8 and 9. The following table indicates the amounts added:

| | |
|---|---|
| Jurkat cells (Control) | 5.33 |
| Jurkat cells + anti CD3 | 27.49 |
| Jurkat cells + anti CD3 + anti FasL neutralization Ab | 12.74 |
| Jurkat cells + anti CD3 + OPG2-Fc 4 ug | 26.24 |
| Jurkat cells + anti CD3 + mFLINT/PG3 3000 ng | 14.68 |
| Jurkat cells + anti CD3 + mFLINT 2000 ng | 17.02 |
| Jurkat cells + anti CD3 + mFLINT 1000 ng | 24.29 |
| Jurkat cells + anti CD3 + mFLINT 500 ng | 27.48 |
| Jurkat cells + anti CD3 + mFLINT 250 ng | 28.93 |
| Jurkat cells + anti CD3 + mFLINT 125 ng | 29.4 |
| Jurkat cells + anti CD3 + mFLINT 62.5 ng | 28.99 |
| Jurkat cells + anti CD3 + mFLINT 31.25 ng | 28.21 |
| Jurkat cells + anti CD3 + mFLINT 15.625 ng | 28.80 |

### EXAMPLE 15

### Measuring the Effect of Human mFLINT on Murine Fasl-Mediated Apoptosis using Mouse T Cell Hybridoma Cells (LTT Cells) (Annexin V Assay)

FLINT was made according to Examples 8 and 9. LTT,2,14,11 cells (LTT cells), see Glasebrook, Eur.J.Immunol. 17: 1561-65 (1987), were used in this Annexin V assay. On the first day, a 96 well plate was coated with anti-CD3 (2C11) at a serial dilutions. On the second day, 100,000 LTT cells were added, in 50 μl of medium per well, along with 50 μl of medium to the control wells and 50 μl of medium containing:

Group 1. solubleFas (sFas)(FasFc, mouse), with a final concentration of 1 μg/ml

Group 2. mFLINT (human), with a final concentration of 1 ug/ml

Group 3. anti-FasL (mouse), with a final concentration of 1 ug/ml.

These then were incubated overnight, at 37° C., in 5% CO₂;

On the following day (Day 3), the cells were collected from each well, washed and labeled with Annexin V and PI, following Flow cytometry analysis.

| | Control % | SFas % | FLINT % | Anti-FasL % |
|---|---|---|---|---|
| | Annexin V | Annexin V | Annexin V | Annexin V |
| Anti CD3 | Positive | Positive | Positive | Positive |
| 1 | 96.42 | 56.66 | 34.41 | 53.46 |
| 0.33 | 94.48 | 47.28 | 35.89 | 39.21 |
| 0.11 | 91.27 | 41.08 | 33.24 | 32.54 |
| 0 | 19.74 | 23.14 | 26.47 | 17.54 |

### EXAMPLE 16

### Measuring the Effect of Human mFLINT on Murine Fasl-Mediated Apoptosis using Mouse T Cell Hybridoma Cells (LTT Cells (Cytotoxicity Assay)

The same steps as in Example 16 were followed on Day 1 and Day 2. On Day 3 20 ul of MTS solution (Promega) was added to the cells which were then incubated at 37° C. for 2 hours. Using a plate reader, the absorbances at 490 nm wavelength were collected.

| Anti CD3 conc. | s-Fas | FLINT | Anti- | |
|---|---|---|---|---|
| (μg/ml) | (1 μg/ml)) | (1 μg/ml) | FasL | Control |
| 0 | 1.774 | 1.691 | 2.01 | 1.534 |
| 0.0014 | 1.968 | 1.923 | 2.134 | 1.614 |
| 0.004 | 1.929 | 1.982 | 2.147 | 1.653 |
| 0.012 | 1.779 | 2.006 | 2.108 | 1.284 |
| 0.037 | 1.777 | 2.006 | 1.988 | 0.834 |
| 0.11 | 1.638 | 1.874 | 1.956 | 0.733 |
| 0.33 | 1.624 | 1.671 | 1.978 | 0.648 |
| 1 | 1.459 | 1.581 | 1.887 | 0.664 |

### EXAMPLE 17

### LIGHT Binding Experiments

To confirm the dot blot binding between LIGHT and mFLINT, 293 cells were transiently transfected with LIGHT expression construct overnight. On the next day, cells were detached and incubated with mFLINT-Flag on ice. The Flag epitope was subsequently detected by anti-Flag conjugated with fluorochrome and the cells population that shows specific binding with mFLINT was detected by flow cytometer. As a control we used vector transfected cells. To make sure that the binding was specific, a competition assay with 10 fold access of untagged mFLINT was performed.

More particularly, 6 well dishes of cells were transfected as above. Both vector and m-LIGHT expressing cells were provided. Cells were detached from the plates by vigorous pipetting with a P100 Pipettor. Then, in PBS/BSA, 0.1%, cells were exposed to one of the following combinations, set forth in a-d.

a. GST/flag, mFLINT/flag, or HVEM at 20 nM;

b. FLINT/flag at 20 nM+GST/flag or mFLINT or HVEM at 200 nM;

c. FLINT/flag at 20 nM+HVEM+anti-human FAS ligand at 200 n-M;

d. anti-human FAS ligand-biotin at 1 μg/ml

Cells were incubated on ice for 30 minutes and washed with PBS/BSA, 0.1%. Cells then were exposed either to anti-human IgG-biotin, 1 μg/ml (for detection of HVEM), or to M2-biotin, 2 μg/ml (for detection of flag conjugates). Cells were incubated on ice for 30 minutes and washed with PBS/BSA, 0.1%. Thereafter, cells were exposed to streptavidin Alexa 488 (SIGMA) at a 1:1000 dilution. Again, cells were incubated on ice for 30 minutes and washed with PBS/BSA, 0.1 1%. Cells were analyzed using a FACSORT flow cytometer (Decton Dickinson) to determine binding.

The cell surface binding assay using flow cytometer-confirmed that peaks shifted only when LIGHT expressing cells were stained with mFLINT-Flag was used (data not shown). There was no shift in the control cells when stained with mFLINT-Flag. The shifted peak was completely reversed to baseline peak when the cells were preincubated with 10 fold excess of non tagged mFLINT thereby preoccupying all the binding sites for mFLINT-Flag.

### EXAMPLE 18

### In vivo Testing of mFLINT for Treatment of Liver Damage

Using mice, a model of liver damage was induced using a modification of the methods set out in Tsuji H., et al, 1997, Infection and Immunity, 65(5).1892-1898. mFLINT was made according to Examples 8 and 9.

Specifically, the activity of the polypeptides of the present invention against acute inflammation and apoptosis was determined using the following procedure. Briefly, BALB/c mice (Harlan) per each experimental group were given intravenous injections (the lateral tail vein) of 6 mg of D(+)-Galactosamine (Sigma, 39F-0539) in 100 μl of PBS (GIBCO-BRL) and 3 μg of Lipopolysaccharide B E. coli 026:B6 (LPS) (Difco, 3920-25-2) in 100 μl of PBS. The LPS was administered, via i.v. injection, 5 minutes after the galactosamine, which was administered i.v. After LPS challenge, the animals were injected intraperitoneally with mFLINT (200 μg), Hamster IgG (500 μg, Cappel, 30926), mAb against murine TNF, TN3-19.12 (500 μg, Sheehan K. C. F. et al J. Immunol. 1989. 142: 3884), and Anti-mouse Fas Ligand (500 μg, PharMingen, M024301) at 0, 2, 4, 6 hour-point respectively. The survival rates of the mice were determined 24 and 48 hours after LPS injection.

In mice challenged with 3 μg of LPS after IP injection of 200 μg mFLINT polypeptide (FLINT) had a positive effect on animal survival. When mFLINT was administered 2 hours post-challenge, 100% of the animals survived; at 4 hours post-challenge, 73% of the animals survived; at 6 hours post-challenge, 60% of the animals survived. In contrast, administration of anti-TNF∝ at 4 hours post-challenge protected only 10% of the animals.

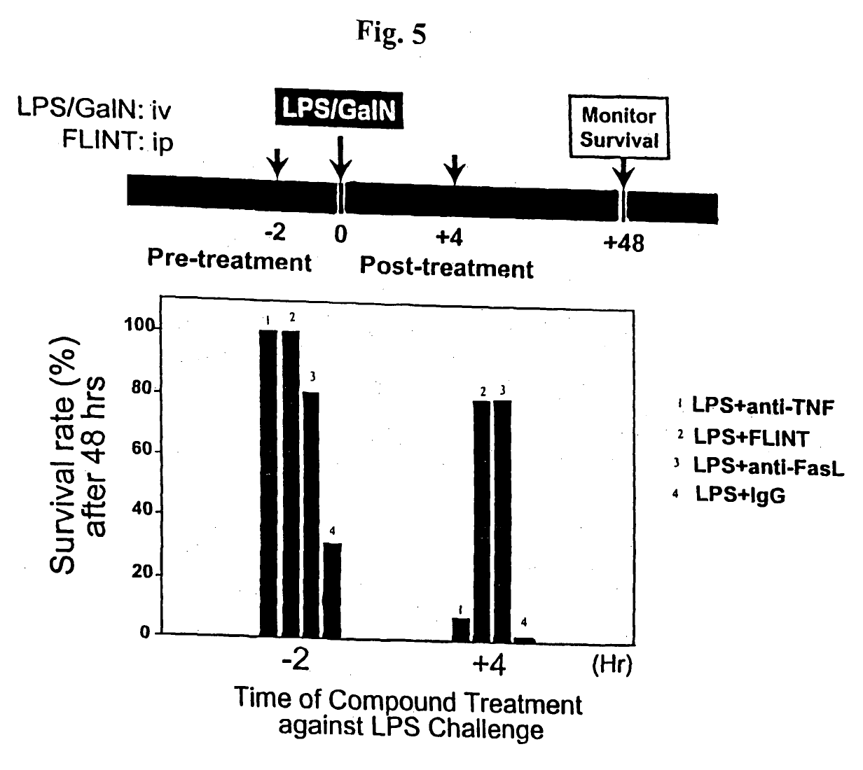

FIG. 5 compares the effects of administering 200 μg mFLINT, i.v., and other molecules before and after challenge with LPS/GalN. When administered 2 hours before LPS/GalN, both anti-TNF and mFLINT treatment resulted in 100% survival of mice. 80% of mice treated with anti-FasL survived, and about 30% of mice treated with IgG survived.

In contrast, when anti-TNF was administered 4 hours after LPS/GalN, less than 10% of the animals-survived at 48 hours. Surprisingly, 80% of the animals survived when treated with mFLINT, at 4 hours after LPS/GalN treatment. Administration of anti-FasL at 4 hours after LPS/GalN treatment also resulted in 80% survival. IgG had essentially no effect on survival, with only about 2% of the animals surviving. Therefore, mFLINT is effective at treating late-stage disease.

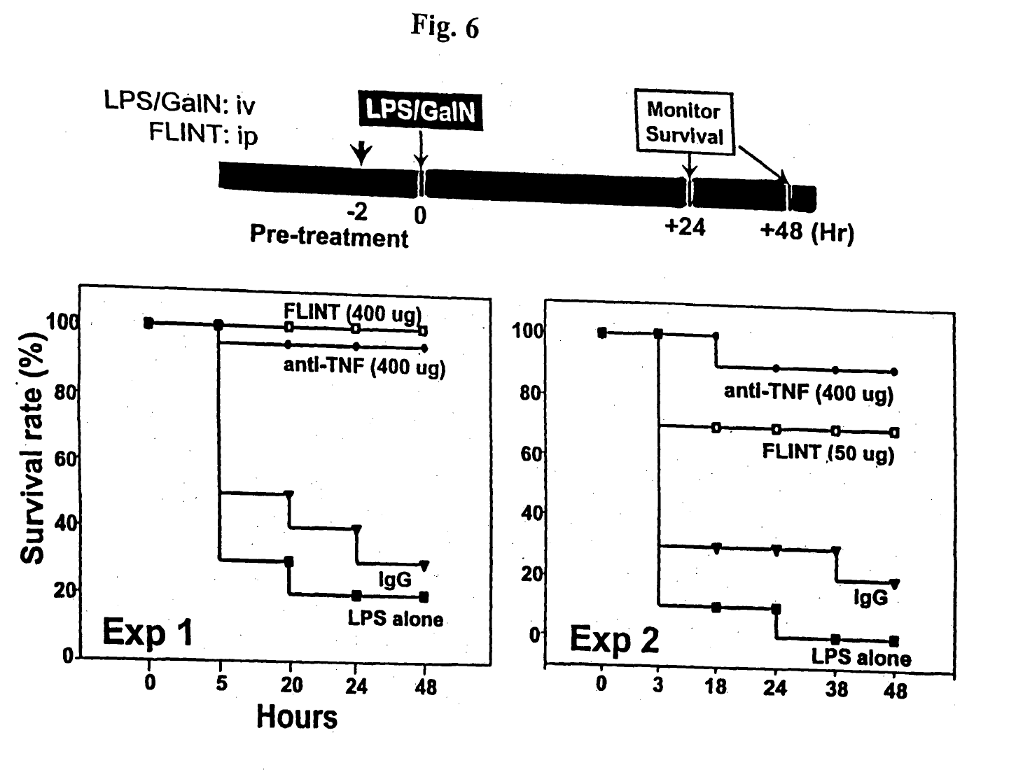

FIG. 6 shows that when 400 ug of mFLINT was administered i.p. to mice, 2 hours before challenge with LPS and GalN, 100% of the animals were alive 48 hours after LPS/GalN dosing. If 400 ug of anti-TNF was given i.p. two hours before LPS/GalN challenge, about 95% of the animals survived to 48 hours. In contrast, only 20% of those animals not treated with mFLINT survived 48 hours. Administration of IgG two hours before LPS/GalN only increased the survival rate to 30%.

FIG. 6 shows that lowering the dose of mFLINT to 50 ug still had a protective effect. 48 hours after LPS/GalN administration, about 70% of the animals survived to 48 hours, compared with 0% survival of non-treated animals.

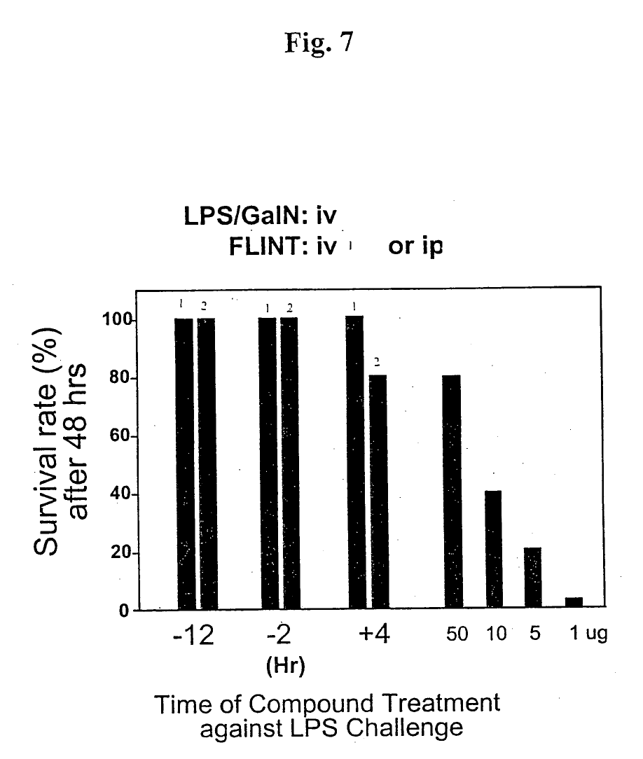

FIG. 7 shows that the effect of 100 μg mFLINT on survival is the same, whether given ip or iv, 12 or 2 hours before LPS/GalN administration. When mFLINT is given 4 hours after LPS/GalN administration, iv administration results in 100% survival, and IP administration results in 80% survival. This Figure also shows the dose/response relationship of the 4-hour post-LPS/GalN dose, given iv. 50 ug gave 80% survival, 10 ug gave 40% survival, 5 ug gave 20% survival and 1 ug gave only about 2% survival.

### EXAMPLE 19

This example demonstrates the usefulness of mFLINT in the treatment of cerebral ischemia, which is clinically relevant in stroke, head trauma and other similar disorders.

Adult male gerbils (70 to 80 g body weight, Charles River Laboratories, Wilmington, Mass.) were anesthetized by i.p. injections of sodium pentobarbital (Nembutal) 40 mg/kg, and additional i.p. injections of 10 mg/kg when necessary to maintain a surgical plane of anesthesia. Animals were placed on a thermostatically controlled heating blanket to maintain body temperature at 37° C. The ventral surface of the neck was exposed, the fur shaved, and the skin cleaned with 2% iodine solution.

After the pre-surgical preparation, a midline incision was made, and the skin opened. The sternohyoid muscles were divided to exposed and isolate the common carotid arteries (CCA) for clamping. Sterilized aneurysm clips (blade with 0.15 mm, closing force ^{˜}10 gm) was secured by means of a sterilized clip applier on both left and right CCA for 5 minutes. The clamps were then removed and the patency of the arteries checked visually. The wound in the neck was closed by surgical suture.

Immediately following the cerebral ischemia procedure and while the gerbil was still unconscious, the fur on the dorsal surface of the head was shaved and the skin cleaned with 2% iodine solution. Under surgical anesthesia, the gerbil's head was secured in a stable position by means of a stereotaxic apparatus (SA) and a midline incision was made to expose the skull. At a position 1 mm lateral and 1 mm posterior to the bregma, as guided by the vernier scale of the SA, the skull was thinned by a dental drill equipped with a drill bit of 0.5 mm in diameter. The thinned area was punctured with a microsyringe equipped with a 27-guage blunt needle inserted 3 mm deep for a bolus injection of 5 ul (0.63 mg/ml )of mFLINT in phosphate buffer saline (PBS). mFLINT was made according to Examples 8 and 9.

After the bolus injection, the syringe needle was exchanged for an infusion cannula [3 mm in length] of a brain infusion assembly connected to an Alzet osmotic pump (Alza Corp., Palo Alto, Calif.) which reservior was placed under the skin on the shoulder of the gerbil. The infusion cannula was anchored on the surface of the skull using dental cement. The wound was closed by surgical suture. The Alzet osmotic pump containing mFLINT solution (0.63 mg/ml) or PBS delivered continuously at a rate of 1 ul/h for 3 days. Gerbils were allowed to survive for 5 days (the surgery day was taken as day zero).

On the fifth day of survival; the gerbils were sacrificed in a CO2 chamber. Thoracotomy was performed for transcardiac perfusion of saline for 3 minutes and formaldehyde for 2 minutes. The brains were removed for histological processing following a standard procedure commonly adapted in the field. Coronal sections were obtained at approx. 1.7 mm posterior to the bregma. After staining with Cresyl violet, the sections were viewed under a microscope at 40× magnification for cell counter quantification of the intact hippocampal neurons along the dorsal CA1 regions (0.5 mm in length) of both hemispheres. Data were analyzed by Student t-Test and the Wilcoxon ranking test.

Results showed that mFLINT had a significant effect on neuronal survival compared to vehicle (p-0.0039 in t-Test; p=0.0037 in Wilcoxon Rank Sums) and was indistinguishable from normal controls.

### EXAMPLE 20

| TREATMENT GROUPS: | Number of Animals |
|---|---|
| CONTROLS | 10 |
| FLINT (50 μg/injection)iv, | 10 |
| days 4-13 | |
| OPG2 (50 μg/injection)iv, | 10 |
| days 4-13 | |
| TOTAL: | 30 mice |

For the experiment, a single cell suspension of B16 melanoma cells was prepared from a brie of donor tumors. Tumor cells (2×10⁶) were implanted subcutaneously in a bind-leg of Male C57B1 mice from Taconic Farms on day 0. Treatment was initiated on day 4. mFLINT or OPG2 was administered by intravenous injection into a tail vein once daily on days 4 through 13 for a total of 10 injections. mFLINT was made according to. Examples 8 and 9. An overview of the protocol is shown above.

Tumor response was monitored by tumor volume measurements as determined by caliper measurements of two dimensions of the tumors on days 4, 8, 11, 17, 21, 25, 30 and 34. Tumor volume was calculated as a hemi-ellipsoid. The animals were weighed on the same schedule described above.

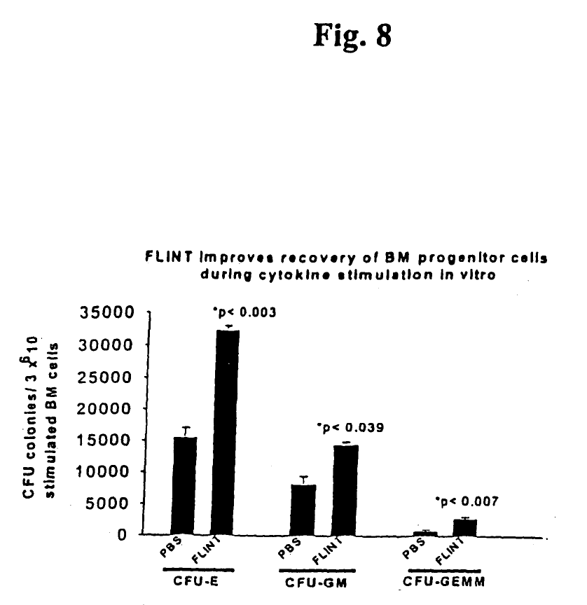

The control tumors reached a volume of 500 mm3 on day 17.4±0.3 and 1000 mm³ on day 21.1±0.3. The tumors of the animals treated with mFLINT reached a volume of 500 mm³ on day 19.3±0.3 and reached 1000 mm³ on day 23.0±0.4. The tumors of the animals treated with OPG2 reached a volume of 500 mm3 on day 185±0.4 and reached 1000 mm³ on day 22.2±0.4. Therefore, the tumor growth delay produced by mFLINT was 1.8 days and the tumor growth delay produced by OPG2 was 1.1 days. These data are shown graphically in FIG. 8. As shown in FIG. 8, tumor volume after 20 days was approximately 730 mm³ in mFLINT-treated mice, but the tumor volume of control mice was approximately 1000 mm³. There was no indication of toxicity from administration of mFLINT or OPG2 as evidenced by weight loss in the animals.

### EXAMPLE 21

Thirty NOD mice 6 weeks of age are purchased from Jackson Laboratories (Bar Harbor, Me.). The mice are housed three to a cage and given free access to food (Purina 5001) and water. After one week of acclimatization, the mice are bled by tail snip, and blood glucose and plasma insulin are measured. Blood glucose is measured by tail snip without anesthetic using a Precision G blood glucose analyzer (Medisense Inc., Bedford, Mass.). Plasma insulin is measured by a RIA kit (Linco Inc., St. Louis, Mo.). The mice are then arbitrarily placed into three groups, control, mFLINT-injected, and OPG2-injected. Following assigning of the mice to groups, body weight and blood glucose are determined once weekly. Beginning at the onset of overt diabetes (blood glucose>200 mg %; ^{˜}14 weeks of age) mice are injected once daily intraperitoneally with either mFLINT (50 μg/day) or OPG2 (50 μg/day) diluted in PBS. Control mice are injected with an equal volume of PBS. After 2 weeks of injection, mice are anesthetized by inhalation anesthesia (carbon dioxide) and blood is removed by cardiac puncture for measurement of glucose and insulin. Additionally, the pancreas is harvested by the Animal Studies Support Team and fixed in zinc-formalin for 24 hours for subsequent histochemical analysis of P cell integrity (hematoxolin and eosin) and immunohistochemical staining for insulin (George Sanduskys laboratory). mFLINT is made according to Examples 8 and 9.

### EXAMPLE 22

This example demonstrates that mFLINT inhibits apoptosis is an in vitro model that mimics ischemia reperfusion injury. These date indicate that mFLINT is useful in preventing and treating such injury.

Ventricular cardiomyocytes were isolated from the hearts of 1-3 day-old neonatal rats by trypsin digestion and non-myocytes were eliminated by pre-plating. Primary cultures were plated in special-coated 96-well plates in serum-free medium.

Hypoxia was induced by incubating cultures in glucose-free and oxygen-free (5% Co₂ and 95% N₂) for 8 hours. Reperfusion was mimicked by a 16 hour incubation in glucose-containing medium under normal oxygen conditions. At the end of this incubation, cardiomyocyte apoptosis was measured by the cytoplasmic nucleosome-associated DNA ELISA method. Test samples were incubated with mFLINT (2 μg/ml or 5 μg/ml) and control samples with Z-YVAD-fmk (50 μM), a commercial caspase inhibitor. Duplicate experiments showed essentially complete inhibition of hypoxia/reperfusion-induced apoptosis. mFLINT was made according to Examples 8 and -9.

To confirm these data and to query whether the observed inhibition was Fas-mediated, cardiomyocyte apoptosis was induced by incubation with soluble FasL, and apoptosis was measured as above. In these experiments, apoptosis was inhibited either by anti-Fas neutralizing antibody (1 μg/ml) or mFLINT (10 μg/ml). These experiments confirmed that mFLINT inhibits apoptosis, and indicated that it does so, at least in part, by inhibiting the FasL-Fas apoptotic pathway.

### EXAMPLE 23

### Murine Osteoclast Differentiation Assay

The co-culture method of Takahashi et al. (Endocrinology 123:2600 1988) was modified as described in Galvin et al. (Endocrinology 137:2457 1996) and used to study the effects of various agents on osteoclast differentiation. mFLINT was made according to Examples 8 and 9.

Male Balb/C mice (4-8 weeks old) were euthanized with CO₂, the femurs removed, and the marrow flushed out of the femurs with growth medium. Bone marrow cells were pelleted by centrifugation at 500×g for 6 min. and resuspended in the growth medium (RPMI 1640 plus 5% heat inactivated fetal bovine-serum and 1% antibiotic-antimycotic solution). The marrow population (5×10⁴ cells/cm²) was seeded in tissue culture dishes in which BALC cells (a stable cell line derived from neonatal mouse calvariae, 1.5×10⁴ cells/cm²) had been plated 2 h prior to addition of bone marrow. The cells were cultured for 7 days in a humidified incubator at 37° C. with 5% CO₂ with medium changes on days 3 and 5. Cultures were treated with or without 10⁻⁸M 1,25-(OH)₂D₃ on days 0, 3, and 5. In addition, -the cells were treated with or without secreted mFLINT protein purified from the conditioned medium of cells transfected with mFLINT gene (FIG. 1). Following 7 days of culture, the cells in 24-well cluster dishes were fixed with formalin (3.7% for 10 min) and then stained for tartrate-resistant acid phosphatase (TRAP) using a modification of the method described by Graves, L and Jilka R L, J Cell Physiology 145:102 1990. The number of osteoclasts (TRAP-positive cells containing 3 or more nuclei) was quantitated. Results are reported in the following Table.

TABLE

| FLINT (ng/ml) | Osteoclasts/well^{a} |
|---|---|
| 0.00 | 145.50 ± 7.33 |
| 0.01 | 40.50 ± 2.39* |
| 0.10 | 65.50 ± 3.33* |
| 1.00 | 97.50 ± 3.10* |
| 10.00 | 170.17 ± 8.26 |
| 100.00 | 335.00 ± 8.90* |

^{a}Each value represents the mean and standard error of 6 wells.
*p < 0.05 compared to control group

### EXAMPLE 24

### Porcine Osteoclast Differentiation Assay

Neonatal pigs (aged 1-5 days) were euthanized with CO₂, the appendages were rinsed with 70% ethanol, the soft tissues were removed, and the humeri, radii, ulnae, femora, tibiae and fibulae were excised. The long bones were placed in ice-cold calcium and magnesium-free Hank's balanced salt solution (CMF-HBSS, Gibco BRL) and cleaned of all soft tissues. The bones were split longitudinally and the endosteal surfaces were scraped to remove both the marrow and trabecular bone. The suspension of trabecular bone particles and marrow cells was agitated by vigorous shaking and passed through a 200 mm and then 100 mm sieve. Cells were centrifuged at 500×g for 10 minutes at 4° C., the pellet was resuspended in CMF-HBSS, and then separated on a Ficoll-Paque gradient (Pharmacia, Piscataway, N.J.). The mononuclear cell fraction from the gradient was washed twice in CMF-HBSS and passed through a 35 mm sieve. The cells were suspended in growth medium consisting of a-MEM (pH 7.2, which was modified to contain 8.3 mM NaHCO₃ (Gibco BRL, Grand Island, N.Y.)), 10% heat-inactivated fetal bovine serum (FBS, Hyclone, Logan, Utah) and 2% antibiotic/antimycotic solution (Gibco BRL, Grand Island, N.Y.) and seeded onto tissue culture dishes at a density of 1×10⁶ cells/cm². A typical marrow cell yield was between 1-2×10⁹ cells/animal, which varied with the size of the animal. The cells were incubated at 37° C. in a humid incubator with 5% CO₂. After 24-48 h, nonadherent cells were removed and seeded in either 24-well cluster dishes at a density of 7.5×10⁵ cells/cm² in growth medium which did or did not contain 10⁻⁸ M 1,25-(OH)₂D₃ (Biomol Plymouth Meeting, Pa.) and “mFLINT protein (obtained as in Examples 8 and 9). Cells were cultured for up to 10 days with medium changes every 48-72 h with growth medium that did or did not contain 1,25-(OH)₂D₃ and mFLINT. Following 5 days of culture, the cells were fixed with formalin (3.7% for 10 min) and then stained for tartrate-resistant acid phosphatase (TRAP) as in Example 6. The number of osteoclasts (TRAP-positive cells containing 3 or more nuclei) was quantitated. Results are reported in the following table.

TABLE

| FLINT (ng/ml) | Osteoclasts/well^{a} |
|---|---|
| 0.00 | 214.83 + 14.22 |
| 0.01 | 68.83 + 6.28* |
| 0.10 | 176.17 + 23.01 |
| 1.00 | 228.50 + 17.26 |
| 10.00 | 228.50 + 29.29 |
| 100.00 | 382.33 + 26.59* |

^{a}Each value represents the mean and standard error of 6 wells.
*p < 0.05 compared to control group

### EXAMPLE 25

### In vitro Treatment with “mFLINT

FLINT was made according to Examples 8 and 9 and used in the following experiments.

A—Bone marrow cells from irradiated mice—This experiment was designed to determine whether mFLINT could improve the recovery of murine hematopoietic progenitor cells from irradiation, by forcing in vitro expansion with hematopoietic cytokines. Mice were injected with 3 mg of 5-flouro-uracil, i.p., and four days later, femurs were removed and flushed to isolate bone marrow (BM) cells. 1×10⁶ BM cells were seeded in each well of a 24 well tray in triplicate in Iscoves modified dulbeccos media (IMDM)+10% FBS and were stimulated for 3 days with the cytokines stem cell factor (SCF) and IL-6, in the presence or absence of mFLINT (1 ug/ml). After a 3-day in vitro expansion period, 5×10³ cells were plated out in triplicate for the CFU assay.

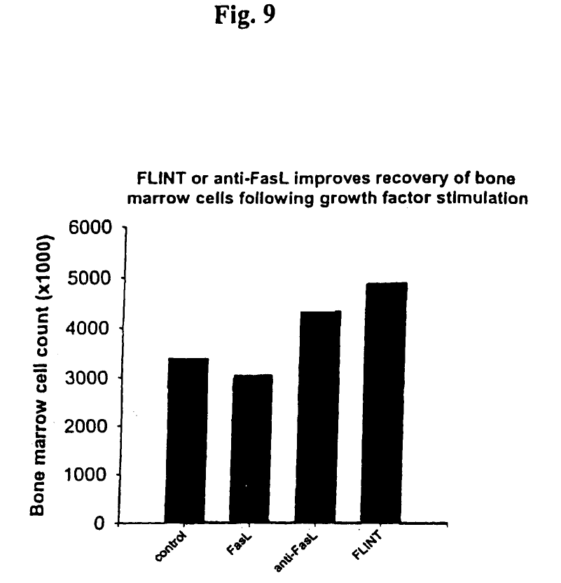

Bone marrow cells incubated with mFLINT had significantly increased numbers of CFUs compared to cells that were not treated with mFLINT. FIG. 9 shows the CFU colonies/3×10⁶ stimulated BM cells. These results suggest that mFLINT protected progenitor cells from apoptosis, and increased the number of viable cells that were able to form colonies. The Figure shows results for the following colony forming units-erythroid cells(CFU-E)(control—15,000 colonies; mFLINT-treated—33,000 colonies)(p<0.003)—granulocyte macrophage cells (CFU-GM) (control—8,000; mFLINT-treated—15,000) (p<0.039); and the more primitive, granulocyte erythroid monocyte megakaryocyte cells (CFU-GEMM)(control—750; mFLINT-treated—3,000) (p<0.007). p numbers show significant differences, as calculated by Student's T-test.

B—Bone marrow cells from mice treated with a chemotherapeutic agent—This experiment was designed to demonstrate that mFLINT or anti-FasL antibody can improve the recovery of murine hematopoietic progenitor cells that have been exposed to a chemotherapeutic agent, following in vitro expansion with hematopoietic cytokines: 2×10⁶ bone marrow cells, from 5-FU treated mice, were seeded in each well of a 24 well tray in triplicate in IMDM media+10% FBS and were stimulated for 3 days with SCF and IL-6, and with one of the following compounds:

1) “mFLINT (1 ug/ml)

2) anti-FasL (1 ug/ml)

3) FasL (0.15 ug/ml)

4) Control—no additions

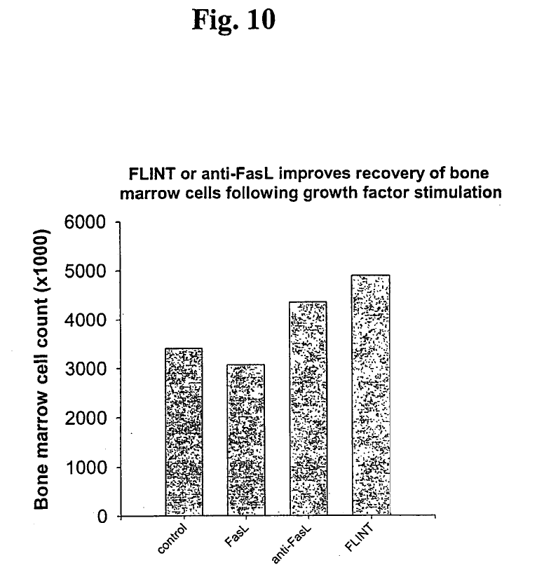

Following a 3 day in vitro expansion period with cytokines, viable cells are counted and used to set up a CFU assay. If mFLINT or anti-FasL antibody protects progenitor cells from apoptosis, one would expect a greater number of colonies in these groups for the CFU assay. Results are shown in FIG. 10, which shows the bone marrow cell count (×1000). The values were: control cells: 3,300; FasL-treated cells: 3,000; anti-FasL-treated cells: 4,200; mFLINT-treated cells: 4,900.

C—CD34+ cells—This experiment is designed to determine whether mFLINT or anti-FasL antibody can improve the recovery of progenitor cells from purified human CD34+ progenitor cells. Purified human CD34+ cells (1×10⁶/ml) are stimulated with 100 U/ml of human IL-6 and 100 ng/ml of human SCF, with or without mFLINT or anti-FasL antibody for 3 days in vitro to expand progenitor cells. Following this expansion and treatment, cells are counted and a CFU assay is conducted.

### EXAMPLE 26

### Transgenic FLINT Mice

A—Preparation of Transgenic Mice

This example demonstrates the construction of transgenic mice expressing FLINT. These animals express significant levels of FLINT, yet demonstrate no adverse effects, which indicates that FLINT has a favorable toxicity profile. These animals are also useful in further delineating useful treatment protocols for the conditions set out above.

Transgene construction. Polymerase chain reaction (PCR) primers were synthesized and used to amplify the fused FLINT-FLAG DNA sequences from plasmid pIG1-FLINTF, described above. The 5′ primer, 5′-GAAGATCTTCTTTGATC AAGGATGGGCTTCTGGACTT-3′, a BglII restriction site and the 3′ primer, 5′-GGACTAGTCCTGATCATCACTTGTCGTCGTCGTCCTT-3′, contained an SpeI restriction enzyme site. These were used to amplify the entire FLINT and FLAG coding region. The amplified 1.1 kb fragment was ligated into the multiple cloning site of plasmid pMTmcs2 generating plasmid pMTmcs2-FLINT (6.7 kb). See Fox et al, Eur. J. Pharmacol. 308:195 (1996).

The FLINT gene fragment was excised from pMTmcs2-FLINT by BglII and SpeI digestion and gel-purified. This fragment then was blunted with Klenow enzyme and ligated into the Klenow-blunted MluI site of plasmid pLIV.7, provided by John Taylor of the J. David Gladstone Institutes. See Fan et al., Proc. Nat'l Acad. Sci. 91:8724 (1994). Resultant plasmid pLIV7-FLINT also contains the apo E gene promoter/5′ flanking region and an hepatic enhancer sequences called the “hepatic control region” (HCR). For microinjection into embryos, a 7.0 kb DNA fragment encompassing the Apo E gene promoter-FLINT/FLAG-HCR fusion gene was excised from plasmid pLIV7-FLINT by digestion with SalI and SpeI and purified by gel electrophoresis and glass bead extraction.

Transgenic animal development. Transgenic mice were generated using established techniques described, for example, by Hogan, B. et al. (1986) MANIPULATING THE MOUSE EMBRYO: A LABORATORY MANUAL, Cold Spring Harbor Laboratory (Cold Spring Harbor, N.Y.), as modified by Fox and Solter, Molec. Cell. Biol. 8: 5470 (1988). Briefly, the 7.0 kb DNA fragment encompassing the Apo E gene promoter-FLINT-HCR fusion gene was microinjected into the male pronuclei of newly fertilized one-cell-stage embryos (zygotes) of the FVB/N strain. The embryos were cultured in vitro overnight to allow development to the two-cell-stage. Two-cell embryos were then transplanted into the oviducts of pseudopregnant CD-1 strain mice to allow development to term. To test for the presence of the transgene in the newborn mice, a small piece of toe was removed from each animal and digested with proteinase K to release the nucleic acids. A sample of the toe extract was subsequently subjected to PCR analysis using human FLINT-specific primers to identify transgene-containing mice. Five founder transgenic mice were identified as containing a FLINT transgene and were designated 6494, 7262, 7353, 7653 and 7659. Each of these founders was bred to produce stable lines of transgenics.

Line 6494 has been extensively characterized for transgene expression and pathology. Broad tissue expression was detected in line 6494 progeny by Northern blot, RT-PCR (TaqMan), Western blot and immunohistochemical analysis and was highest in the liver and kidney. High levels of FLINT protein were detected in the circulation of line 6494 mice by ELISA assay; founder level=490 ng/ml; progeny levels ranged from 285-1360 ng/ml (n=6). No endogenous FLINT was detected by these analyses. No significant histopathologic findings were detected in 5 week or 8 week old 6494 progeny. Preliminary blood chemistry analysis suggested that triglyceride levels may be elevated in 6494 mice. The animals had no observable abnormalities.

B—Protection of mice from irradiation—This experiment is designed to evaluate whether transgenic FLINT mice are protected from the adverse effects of radiation. Lethally irradiate 18 transgenic and 18 non-transgenic mice with 850 cGy. Give no bone marrow transplant to 8 of the 18 mice per group. Of these 8 mice, use 5 as a control for radiation induced death and 3 for histological analysis of the intestinal mucosa and bone marrow compartments at 3 days post-irradiation.

Transplant the remaining 10 mice per group with 3×10⁴ bone marrow cells from normal non-transgenic donors. Since the liver is not damaged by irradiation, the FLINT transgenic mice should still be able to produce FLINT protein systemically. This bone marrow cell dose typically results in about 50% survival at 30 days for wild type animals that have been irradiated as described herein. Obtain 50-100 μl of blood by tail bleed on days 7, 15, 21, and 30 days post-transplant and perform hematological analysis to monitor recovery of peripheral blood (n=10 mice per group; total of 20 mice). The hematological analysis will include a white blood cell count (WBC), red blood cell count (RBC), hematocrit, and blood smear microscopy to determine numbers of lymphocytes, neutrophils, eosinophils, platelets, mast cells and monocytes in the blood. Measure survival of mice and recovery of peripheral blood cells.

It is expected that, because FLINT prevents apoptosis of expanding progenitor cells and/or enhances recovery of intestinal epithelium from radiation-induced damage, there will be a difference between the two groups in survival, histology of gut epithelium and bone marrow, and recovery of peripheral blood cells.

C-Protection of mice from chemotherapy—This experiment is designed to determine the effects of combining FLINT and GM-CSF administration on the recovery of progenitor cells from myelosuppression that is induced by sub-lethal irradiation and chemotherapy. In particular, this experiment examines whether white blood cell recovery, from a combination of chemotherapy and sublethal irradiation, is improved in transgenic FLINT mice. On example of such a chemotherapy regimen is treatment with carboplatin.

Administer 500 cGY of irradiation 4 hours following a single intraperitoneal injection of carboplatin (1.2 mg/mouse) to 15 transgenic and 15 control mice. This regimen induces severe myelosuppression with prolonged thrombocytopenia and severe anemia. Leonard et al., Blood, 83: 1499 (1994). Every day for 12 days, beginning 24 hours after irradiation, 10 μg/kg body weight of recombinant murine rmGM-CSF is injected i.p. to promote recovery of WBC count. Mayer et al., J. Inf. Diseases 163: 584 (1991), Gamba-Vitalo et al., 1991, Blood Cells 17: 193 (1991). Blood is collected by tail bleed every 7 days up to day 30 and a complete hematological analysis is conducted. The hematological analysis will include a white blood cell count (WBC), red blood cell count (RBC), hematocrit, and blood smear microscopy to determine numbers of lymphocytes, neutrophils, eosinophils, platelets, mast cells and monocytes in the blood. On days 12 and 20, 3 mice from each group are sacrificed. From these mice, spleen cellularity and bone marrow cellularity are analyzed. Also, the bone marrow cells are pooled and used to set up CFU assays to examine progenitor content.

D—FLINT effects on peripheral mobilization of progenitor cells—This experiment is designed to test for testing peripheral mobilization of progenitor cells. To test for peripheral mobilization of progenitor cells, FLINT transgenic and control mice are treated with 3 mg of 5-FU i.p. and 4 days later, either CFU assays will be set up from isolated bone marrow cells, or 100 ng of GM-CSF will be administered i.p. every day for 3 days followed by determination of bone marrow cellularity and progenitor content by CFU assays. The protocols for transplantation are described above in Example B. Applications for gene, therapy would be to improve the recovery of progenitor cells following stimulation with cytokines either in vivo, or in vitro, for transduction with retrovirus, as described above.

We claim:

1. A method of treating an individual suffering from acute liver failure, comprising administration of a therapeutically amount of mFLINT protein to said individual.

2. A method of treating an individual suffering from inflammation of the liver, comprising administration of a therapeutically amount of mFLINT protein to said individual.

3. A method of treating an individual suffering from abnormal hepatocyte apoptosis, comprising administration of a therapeutically amount of mFLINT protein to said individual.

4. A method of treating an individual suffering from sepsis, comprising administration of a therapeutically amount of mFLINT protein to said individual.

5. A method of treating an individual suffering from a disorder associated with inflammation, comprising administration of a therapeutically amount of mFLINT protein to said individual.

6. A method of treating an individual suffering from hepatitis, comprising administration of a therapeutically effective amount of mFLINT protein to said individual.

7. A method of treating an individual suffering from abnormal apoptosis, comprising administration of a therapeutically effective amount of mFLINT protein to said individual.

8. A method of treating an individual suffering from an ischemia-associated injury or disorder, comprising administration of a therapeutically effective amount of mFLINT protein to said individual.

9. A method according to claim 8, wherein said injury or disorder is associated with hypercoagulation.

10. A method according to claim 8, further comprising administration of an agent selected from the group selected from thrombolytic and antithrombotic agents.

11. A method according to claim 10, wherein said antithrombotic agent is activated protein C.

12. A method of treating an individual suffering from a reperfusion-associated injury or disorder, comprising administration of a therapeutically effective amount of mFLINT protein to said individual.

13. A method of preventing damage to a cardiac myocyte in an individual that has suffered from abnormal myocardial ischemia, comprising administration of a therapeutically effective amount of mFLINT protein to said individual.

14. A method of treating an individual suffering from Type I diabetes, comprising administration of a therapeutically amount of mFLINT protein to said individual.

15. A method of treating an individual suffering from cancer, comprising administration of a therapeutically effective amount of mFLINT protein to said individual.

16. A method of treating damage to an innocent bystander tissue that is induced by a chemotherapeutic agent or therapeutic irradiation, in an individual treated with said agent or irradiation, comprising administration of a therapeutically effective amount of mFLINT to said individual.

17. A method according to claim 16, wherein said tissue is bone marrow.

18. A method according to claim 16, wherein said tissue is the intestinal epithelium.

19. A method according to claim 18, wherein said epithelium is in the oral cavity.

20. A method of treating hematopoietic progenitor cells that have been exposed to therapeutic radiation or chemotherapy, comprising-administering mFLINT to said cells.

21. A method of promoting the growth or differentiation of a hematopoietic progenitor cell, comprising administering mFLINT to said cell.

22. A method of promoting the growth or differentiation of a CD34+ cell, comprising administering mFLINT to said cell.

23. A method for treating cancer, comprising treating bone marrow cells in vitro with mFLINT, and administering said cells to said patient, wherein said administration occurs after said patient has been treated with therapeutic irradiation or chemotherapy.

24. A method according to claim 23, wherein said cells are from said patient.

25. A method according to claim 23, wherein said cells are from an individual other than said patient.

26. A method of treating cell damage in a patient who receives therapeutic irradiation or chemotherapy, comprising administering to said patient, a therapeutically effective amount of mFLINT with said irradiation or chemotherapy.

27. A method according to claim 26, wherein said cell is an intestinal epithelial cell, a hematopoietic progenitor cell, or a peripheral blood cell.

28. A method of treating aplastic anemia, comprising administering a therapeutically effective amount of mFLINT to a patient suffering from aplastic anemia.

29. A method of treating a myelodysplastic syndrome, comprising administering a therapeutically effective amount of mFLINT to a patient suffering from said syndrome.

30. A method of treating a pancytopenic condition, comprising administering a therapeutically effective amount of mFLINT to a patient suffering from said condition.

31. An isolated nucleic acid molecule having the sequence of FIG. 1.

32. An isolated nucleic acid molecule having the sequence of FIG. 3.

33. An isolated polypeptide having the sequence of FIG. 1.

34. An isolated polypeptide having the sequence of FIG. 3.

35. A mouse comprising a transgene having the sequence of FIG. 1.


SEQUENCE LISTING (13 sequences)

SEQ ID NO: 1 - DNA, 900 bp, homo sapiens
  CDS (1)..(900)
atgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcac

SEQ ID NO: 2 - protein, 300 aa, homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 3 - DNA, 936 bp, homo sapiens
  CDS (25)..(924)
gctctccctgctccagcaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcattgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctgggctccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccc

SEQ ID NO: 4 - protein, 300 aa, homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALIVPGSSSHDTLCTSCTGFPLSTRVPGAEECERAVIDFVAFQDISIKRLQRLLQALEAPEGWAPTPRAGRAALQLKLRRRLTELLGAQDGALLVRLLQALRVARMPGLERSVRERFLPVH

SEQ ID NO: 5 - DNA, 813 bp, homo sapiens
  CDS (1)..(813)
gtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcac

SEQ ID NO: 6 - protein, 271 aa, homo sapiens
VAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 7 - DNA, 825 bp, homo sapiens
  CDS (1)..(813)
gtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctgggctccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccc

SEQ ID NO: 8 - protein, 271 aa, homo sapiens
VAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWAPTPRAGRAALQLKLRRRLTELLGAQDGALLVRLLQALRVARMPGLERSVRERFLPVH

SEQ ID NO: 9 - protein, 24 aa, artificial
  primer
ASPTYRLYSASPASPASPASPLYS

SEQ ID NO: 10 - DNA, 59 bp, artificial
  primer
tagggctgatcaaggatgggcttctggacttgggcggcccctccgcaggcggaccgggg

SEQ ID NO: 11 - DNA, 66 bp, artificial
  primer
aggggggcggccgctgatcatcacttgtcgtcgtcgtccttgtagtcgtgcacagggaggaagcgc

SEQ ID NO: 12 - DNA, 37 bp, artificial
  primer
gaagatcttctttgatcaaggatgggcttctggactt

SEQ ID NO: 13 - DNA, 37 bp, artificial
  primer
ggactagtcctgatcatcacttgtcgtcgtcgtcctt